(19) United States
(12) Patent Application Publication
(10) Pub. No.: US 2021/0236006 A1
(43) Pub. Date: Aug. 5, 2021

(54) OPTICAL MEASURING DEVICE

(71) Applicant: Panasonic Intellectual Property Management Co., Ltd., Osaka (JP)

(72) Inventors: TASUKU NAKAMURA, Osaka (JP); TAKAMASA ANDO, Osaka (JP); KENJI NARUMI, Osaka (JP); TSUGUHIRO KORENAGA, Osaka (JP)

(21) Appl. No.: 17/235,064

(22) Filed: Apr. 20, 2021

Related U.S. Application Data
(63) Continuation of application No. PCT/JP2019/043914, filed on Nov. 8, 2019.

(30) Foreign Application Priority Data
Dec. 10, 2018 (JP) 2018-231038

(51) Int. Cl. A61B 5/026; G01F 1/66

(52) U.S. Cl. CPC A61B 5/0261; A61B 2560/0223; G01F 1/661

(57) ABSTRACT

An optical measuring device includes a light source, a photodetector, a control circuit, and a signal processing circuit. The control circuit causes the light source to emit first and second light pulses with which a target of measurement is irradiated, causes the photodetector to detect a first portion of a first reflected light pulse in a first period having a first time length and output a first signal representing an amount of light of the first portion, and causes the photodetector to detect a second portion of a second reflected light pulse in a second period having a second time length and output a second signal representing an amount of light of the second portion. The signal processing circuit generates, based on a fluctuation in the first signal and a fluctuation in the second signal, information indicating a fluctuation in internal state of the target of measurement.

### BACKGROUND

### 1. Technical Field

The present disclosure relates to an optical measuring device.

### 2. Description of the Related Art

Conventionally, there has been known a method for measuring a change in biological information through photoirradiation. For example, Japanese Unexamined Patent Application Publication No. 9-019408 discloses a biological optical measuring device that measures a blood flow change in a brain with a photoirradiator and a photoreceiver placed on the head of a subject. Japanese Unexamined Patent Application Publication No. 2008-284165 discloses a biological information acquisition device that acquires information on a blood flow distribution or blood flow rate of the head of a driver through the use of an infrared radiation unit mounted in the headrest of a driver's seat. Japanese Unexamined Patent Application Publication No. 2003-337102 discloses a biological activity measuring device that measures, in a noncontact manner, information indicating a biological activity of a subject. Japanese Unexamined Patent Application Publication No. 2017-009584 discloses an imaging device that is capable of measuring internal information on a physical object without contact with the physical object and with a reduction in noise caused by a reflection component from a surface of the physical object.

### SUMMARY

In one general aspect, the techniques disclosed here feature an optical measuring device including: a light source that emits light pulses with which a target of measurement is irradiated; a photodetector that detects at least a portion of each of reflected light pulses returning from the target of measurement; a control circuit that controls the light source and the photodetector; and a signal processing circuit that processes a signal outputted from the photodetector. The light pulses include a first light pulse and a second light pulse. The reflected light pulses include a first reflected light pulse attributed to the first light pulse and a second reflected light pulse attributed to the second light pulse. The control circuit causes the light source to emit the first light pulse and the second light pulse at different timings, respectively. The control circuit causes the photodetector to detect a first portion of the first reflected light pulse in a first period having a first time length and output a first signal representing an amount of light of the first portion, the first period starting from a first time point during a first fall time that is a period from a start to an end of a decrease in intensity of the first reflected light pulse. The control circuit causes the photodetector to detect a second portion of the second reflected light pulse in a second period having a second time length and output a second signal representing an amount of light of the second portion, the second period starting from a second time point during a second fall time that is a period from a start to an end of a decrease in intensity of the second reflected light pulse. A time interval from a start of the first fall time to the first time point is different from a time interval from a start of the second fall time to the second time point. The control circuit executes first control more than once, the first control including causing the light source to emit the first light pulse, causing the photodetector to detect the first reflected light pulse, and causing the photodetector to output the first signal. The control circuit executes second control more than once, the second control including causing the light source to emit the second light pulse, causing the photodetector to detect the second reflected light pulse, and causing the photodetector to output the second signal. The signal processing circuit generates, based on a fluctuation in the first signal and a fluctuation in the second signal, information indicating a fluctuation in internal state of the target of measurement.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a diagram schematically showing an example of an optical measuring device;

FIG. 1B is a diagram showing examples of time variations in the intensity of light that arrives at a photodetector;

FIG. 1C is a diagram whose horizontal axis represents the duration of an input light pulse and whose vertical axis represents the amount of light detected by the photodetector;

FIG. 1D is a diagram schematically showing an example configuration of one pixel of the photodetector;

FIG. 1E is a diagram showing an example configuration of the photodetector;

FIG. 1F is a diagram showing an example of operation within one frame;

FIG. 1G is a flow chart schematically showing an operation carried out by a control circuit;

FIG. 2 is a diagram for explaining a method for detecting an internally-scattered component of a light pulse;

FIG. 3A is a diagram schematically showing an example of a timing chart in a case where a surface-reflected component is detected;

FIG. 3B is a diagram schematically showing an example of a timing chart in a case where an internally-scattered component is detected;

FIG. 4 is a diagram for explaining a method for determining an appropriate shutter timing according to distance to a physical object;

FIG. 5 is a flow chart showing an example of an operation of adjusting the shutter timing according to distance to a physical object;

FIG. 6A is a diagram schematically showing an example of a method for detecting a change in cerebral blood flow rate;

FIG. 6B is a diagram schematically showing an example of a method for simultaneously performing measurements at a plurality of places within a target part of a user;

FIG. 7A is a diagram schematically showing an example of a region that is irradiated with light;

FIG. 7B is a diagram schematically showing a change in measurement result attributed to a lateral motion of the head of the user;

FIG. 8A is a diagram schematically showing an example of a rear-end component of a reflected light pulse that is detected in a case where the target part of the user is at a predetermined distance from the device;

FIG. 8B is a diagram schematically showing an example of a rear-end component of a reflected light pulse that is detected in a case where the target part of the user has moved nearer to the device during measurement;

FIG. 9 is a diagram explaining the principle of measurement by an exemplary embodiment;

FIG. 10 is a flow chart showing an example of operation of an optical measuring device according to an exemplary embodiment;

FIG. 11 is a flow chart showing an example of operation of the optical measuring device before the start of a measurement;

FIG. 12 is a flow chart showing another example of operation of the optical measuring device;

FIG. 13 is another diagram explaining the principle of measurement;

FIG. 14 is a flow chart showing still another example of operation of the optical measuring device; and

FIG. 15 is a diagram schematically showing an example of acquisition of cerebral blood flow information on a user sitting on a seat in an automobile.

### DETAILED DESCRIPTION

### Underlying Knowledge Forming Basis of the Present Disclosure

The device described in Japanese Unexamined Patent Application Publication No. 9-019408 measures a change in blood flow kinetic change in a brain with the photoirradiator and the photoreceiver in contact with the head of a subject. The device described in Japanese Unexamined Patent Application Publication No. 2008-284165 acquires a blood flow distribution or blood flow rate of the head of a driver with the infrared radiation unit in proximity to the head of a driver. Either device may cause the subject or the driver to feel stressed by a sense of being constrained.

The devices described in Japanese Unexamined Patent Application Publication No. 2003-337102 and Japanese Unexamined Patent Application Publication No. 2017-009584 can measure information on the biological interior of a user in a noncontact manner by emitting light from a light source toward a target part of the user and detecting reflected light from the target part with a photodetector. Noncontact measurement does not create stress caused by a sense of being constrained. However, in noncontact measurement, a change in distance between a target part of a user and the photodetector leads to a change in timing of incidence upon the photodetector of reflected light from the target part. For this reason, making a measurement without consideration of such a change in timing may result in a decrease in measurement accuracy.

Based on the foregoing consideration, the present inventors have conceived of an optical measuring device according to the following items.

### Item 1

An optical measuring device according to a first item includes a light source that emits light pulses with which a target of measurement is irradiated, a photodetector that detects at least a portion of each of reflected light pulses returning from the target of measurement, a control circuit that controls the light source and the photodetector, and a signal processing circuit that processes a signal outputted from the photodetector. The light pulses include a first light pulse and a second light pulse, and the reflected light pulses include a first reflected light pulse attributed to the first light pulse and a second reflected light pulse attributed to the second light pulse. The control circuit causes the light source to emit the first light pulse and the second light pulse at different timings, respectively, causes the photodetector to detect a first portion of the first reflected light pulse in a first period having a first time length and output a first signal representing an amount of light of the first portion, the first period starting from a first time point during a first fall time that is a period from a start to an end of a decrease in intensity of the first reflected light pulse, and causes the photodetector to detect a second portion of the second reflected light pulse in a second period having a second time length and output a second signal representing an amount of light of the second portion, the second period starting from a second time point during a second fall time that is a period from a start to an end of a decrease in intensity of the second reflected light pulse. A time interval from a start of the first fall time to the first time point is different from a time interval from a start of the second fall time to the second time point. The control circuit executes first control more than once, the first control including causing the light source to emit the first light pulse, causing the photodetector to detect the first reflected light pulse, and causing the photodetector to output the first signal. The control circuit executes second control more than once, the second control including causing the light source to emit the second light pulse, causing the photodetector to detect the second reflected light pulse, and causing the photodetector to output the second signal. The signal processing circuit generates, based on a fluctuation in the first signal and a fluctuation in the second signal, information indicating a fluctuation in internal state of the target of measurement.

A technology of the present disclosure makes it possible to acquire internal information in a noncontact manner even in the case of a change in relative position of a target of measurement and a measuring device during measurement.

### Item 2

In the optical measuring device according to the first item, the first time length and the second time length may be identical to each other.

### Item 3

In the optical measuring device according to the second item, the first time point may precede a third time point at which a value of J(t)=|I(t+δt)−I(t)|/I(t) reaches its maximum in the first fall time, the second time point may follow a fourth time point at which the value of J(t) reaches its maximum in the second fall time, t may be a time to start to detect the first reflected light pulse or the second reflected light pulse, δt may be a very short time, and I(t) may be an amount obtained by integrating an amount of light of the first reflected light pulse detected in the first period or an amount obtained by integrating an amount of light of the second reflected light pulse detected in the second period.

### Item 4

In the optical measuring device according to any of the first to third items, the signal processing circuit may generate the information based on a fluctuation in ratio between the first signal and the second signal.

### Item 5

In the optical measuring device according to any of the first to fourth items, when the internal state of the target of measurement is constant, a value of a ratio between the first signal and the second signal in a case where a distance between the target of measurement and the photodetector is a first distance may be substantially equal to a value of the ratio in a case where the distance between the target of measurement and the photodetector is a second distance that is different from the first distance.

### Item 6

In the optical measuring device according to any of the first to fifth items, the target of measurement may be a living organism, and the information may indicate a fluctuation in amount of blood flow of the target of measurement.

### Item 7

In the optical measuring device according to the sixth item, the blood flow may be cerebral blood flow of the living organism.

### Item 8

In the optical measuring device according to any of the first to seventh items, the control circuit may cause the light source and the photodetector to execute a calibration operation of adjusting the first time point and the second time point, in the calibration operation, the control circuit may cause the light source to emit third light pulses and cause the photodetector to detect third reflected light pulses attributed to the third light pulses, the third reflected light pulses being detected with a very short time shift in time lag between a start of a decrease in intensity of each of the third reflected light pulse and a start of detection, the third reflected light pulses may each be detected in a period having a third time length, and the first time length, the second time length, and the third time length may be identical to one another.

### Item 9

An optical measuring device according to an item 9 includes a light source that emits a first light pulse with which a target of measurement is irradiated, a photodetector that detects at least a portion of a first reflected light pulse returning from the target of measurement due to the first light pulse, a control circuit that controls the light source and the photodetector; and a signal processing circuit that processes a signal outputted from the photodetector. The control circuit causes the light source to emit the first light pulse, causes the photodetector to detect a first portion of the first reflected light pulse in a first period having a first time length and output a first signal representing an amount of light of the first portion, the first period starting from a first time point during a fall time that is a period from a start to an end of a decrease in intensity of the first reflected light pulse, and causes the photodetector to detect a second portion of the first reflected light pulse in a second period having a second time length and output a second signal representing an amount of light of the second portion, the second period starting from a second time point during the fall time. A time interval from a start of the fall time to the first time point is different from a time interval from the start of the fall time to the second time point. The control circuit executes control more than once, the control including causing the light source to emit the first light pulse, causing the photodetector to detect the first reflected light pulse, and causing the photodetector to output the first signal and the second signal. The signal processing circuit generates, based on a fluctuation in the first signal and a fluctuation in the second signal, information indicating a fluctuation in internal state of the target of measurement.

### Item 10

In the optical measuring device according to the ninth item, the first time length and the second time length may be identical to each other.

### Item 11

In the optical measuring device according to the tenth item, the first time point may precede a third time point at which a value of J(t)=|I(t+δt)−I(t)|/I(t) reaches its maximum in the fall time, the second time point may follow the third time point, t may be a time to start to detect the first reflected light pulse, δt may be a very short time, and I(t) may be an amount obtained by integrating an amount of light of the first reflected light pulse detected in the first period.

### Item 12

In the optical measuring device according to any of the ninth to eleventh items, the signal processing circuit may generate the information based on a fluctuation in ratio between the first signal and the second signal.

### Item 13

In the optical measuring device according to any of the ninth to twelfth items, when the internal state of the target of measurement is constant, a value of a ratio between the first signal and the second signal in a case where a distance between the target of measurement and the photodetector is a first distance may be substantially equal to a value of the ratio in a case where the distance between the target of measurement and the photodetector is a second distance that is different from the first distance.

### Item 14

In the optical measuring device according to any of the ninth to thirteenth items, the target of measurement may be a living organism, and the information may indicate a fluctuation in amount of blood flow of the target of measurement.

### Item 15

In the optical measuring device according to the fourteenth item, the blood flow may be cerebral blood flow of the living organism.

### Item 16

In the optical measuring device according to any of the first to fifteenth items, the control circuit may cause the light source and the photodetector to execute a calibration operation of adjusting the first time point and the second time point, in the calibration operation, the control circuit may cause the light source to emit second light pulses and cause the photodetector to detect second reflected light pulses attributed to the second light pulses, the second reflected light pulses being detected with a very short time shift in time lag between a start of a decrease in intensity of each of the second reflected light pulse and a start of detection, the second reflected light pulses may each be detected in a period having a third time length, and the first time length, the second time length, and the third time length may be identical to one another.

The embodiment to be described below illustrates general or specific examples. The numerical values, shapes, materials, constituent elements, locations of placement, and the like that are shown in the following embodiment are mere examples and are not intended to limit the present disclosure. Further, those of the constituent elements according to the following embodiment which are not recited in an independent claim indicating a most generic concept are described as optional constituent elements.

In the present disclosure, all or some of the circuits, units, devices, members, or sections or all or some of the functional blocks in the block diagrams may be implemented as one or more of electronic circuits including, but not limited to, a semiconductor device, a semiconductor integrated circuit (IC), or an LSI (large scale integration). The LSI or IC can be integrated into one chip, or also can be a combination of multiple chips. For example, functional blocks other than a memory may be integrated into one chip. The name used here is LSI or IC, but it may also be called system LSI, VLSI (very large scale integration), or ULSI (ultra large scale integration) depending on the degree of integration. A Field Programmable Gate Array (FPGA) that can be programmed after manufacturing an LSI or a reconfigurable logic device that allows reconfiguration of the connection or setup of circuit cells inside the LSI can be used for the same purpose.

Further, it is also possible that all or some of the functions or operations of the circuits, units, devices, members, or sections are implemented by executing software. In such a case, the software is recorded on one or more non-transitory recording media such as a ROM, an optical disk, or a hard disk drive, and when the software is executed by a processor, the software causes the processor together with peripheral devices to execute the functions specified in the software. A system or device may include such one or more non-transitory recording media on which the software is recorded and a processor together with necessary hardware devices such as an interface.

In the following, an embodiment is specifically described with reference to the drawings. In the following description, identical or similar constituent elements are given the same reference signs.

### Embodiment

### 1. Optical Measuring Device 100

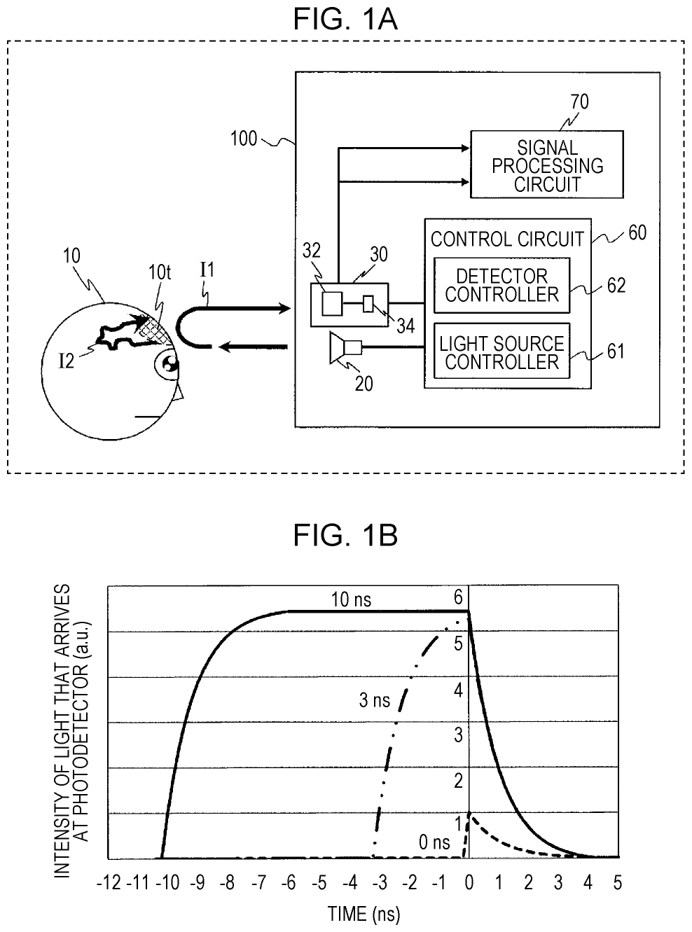

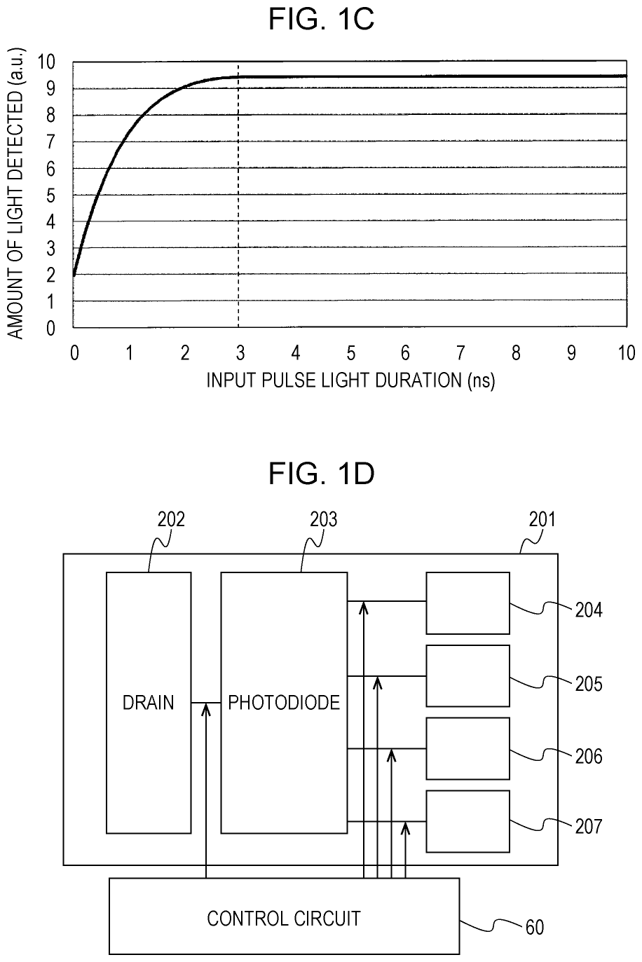

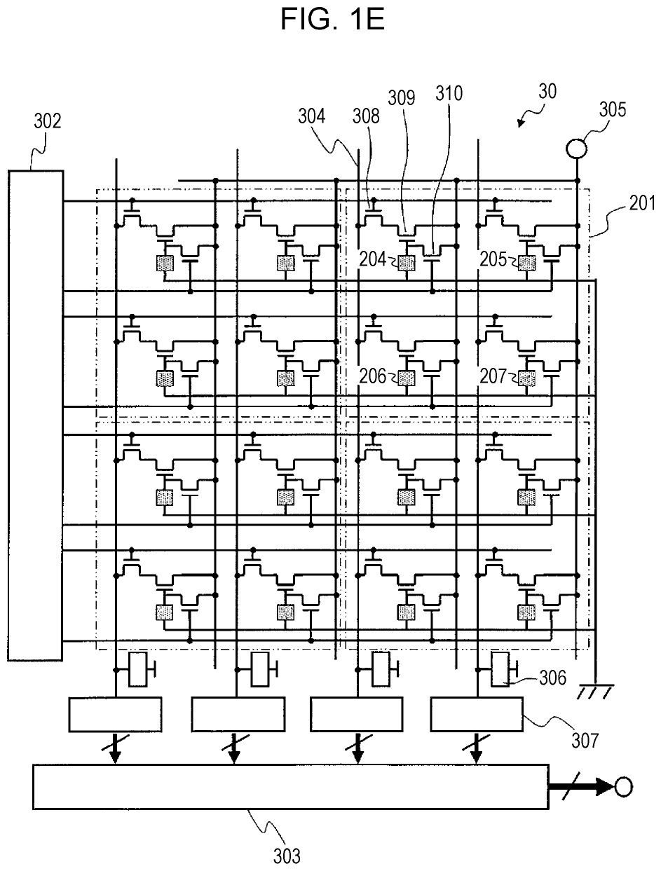

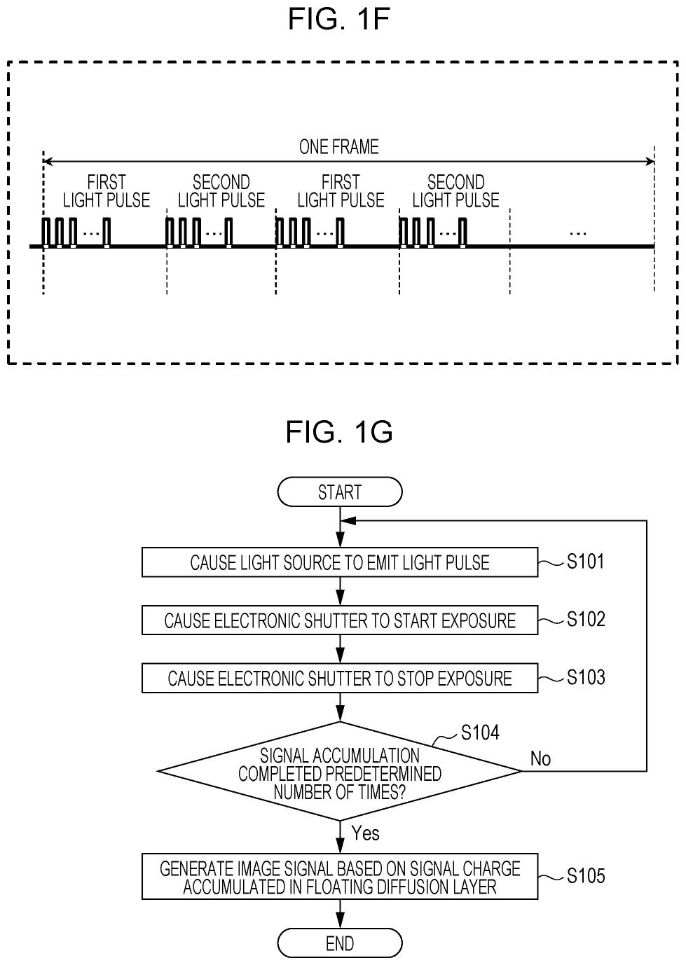

A configuration of an optical measuring device 100 according to an exemplary embodiment of the present disclosure is described with reference to FIGS. 1A to 3B.

FIG. 1A is a diagram schematically showing an example of the optical measuring device 100 according to the present embodiment.

The optical measuring device 100 includes a light source 20, a photodetector 30, a control circuit 60, and a signal processing circuit 70. The photodetector 30 according to the present embodiment is an image sensor that acquires a two-dimensional image. The photodetector 30 is not limited to an image sensor that acquires a two-dimensional image, but may be an image sensor that acquires a one-dimensional image. Depending on applications, the photodetector 30 may be a sensor including a single photoelectric conversion element.

The light source 20 emits a light pulse with which a target part 10t of a user 10, i.e. a target of measurement, is irradiated. The photodetector 30 detects the amount of light of at least a portion of a reflected light pulse returning from the target part 10t of the user 10 and outputs a signal representing the amount of light. The control circuit 60 controls the light source 20 and the photodetector 30. The signal processing circuit 70 processes a signal outputted from the photodetector 30.

In the present embodiment, the control circuit 60 includes a light source controller 61 that controls the light source 20 and a detector controller 62 that controls the photodetector 30. The light source controller 61 controls the intensity, pulse duration, emission timing, and/or wavelength of a light pulse that is emitted from the light source 20. The detector controller 62 controls the timing of signal accumulation in each pixel of the photodetector 30.

The term “biological information” herein means a measurable quantity of a living organism. Examples of the biological information include various quantities such as a blood flow rate, blood pressure, a heart rate, a pulse rate, a respiratory rate, body temperature, brain waves, the concentration of oxyhemoglobin in blood, the concentration of deoxyhemoglobin in blood, a blood oxygen saturation level, and a reflectance spectrum of skin. Some of the biological information may be referred to as “vital signs”. The following describes each constituent element of the optical measuring device 100.

### 1-1. Light Source 20

The light source 20 emits light toward the target part 10t of the user 10. The target part 10t is for example the head of the user 10 or, more specifically, may be the forehead of the user 10. In a case where brain activity information is not utilized, the target part 10t of the user 10 may for example be an arm, a torso, or a foot. Light emitted from the light source 20 and having arrived at the user 10 is split into a surface-reflected component I1 that is reflected off a surface of the user 10 and an internally-scattered component I2 that is scattered within the user 10. The internally-scattered component I2 is a component that is reflected or scattered once or multiply scattered within a living organism. In a case where light is emitted toward the head of the user 10, the internally-scattered component I2 refers to a component that arrives at a site 8 to 16 mm deep from the surface of the head of the user 10, e.g. the brain, and returns to the optical measuring device 100. The surface-reflected component I1 includes three components, namely a directly-reflected component, a diffusedly-reflected component, and a scattered reflected component. The directly-reflected component is a reflection component whose angle of incidence and angle of reflection are equal. The diffusedly-reflected component is a component that is reflected while being diffused by asperities on the surface. The scattered reflected component is a component that is reflected while being scattered by internal tissue near the surface. In a case where light is emitted toward the head of the user 10, the scattered reflected component is a component that is reflected while being scattered within the epidermis. The surface-reflected component I1, which is reflected off the surface of the user 10, may include these three components. The surface-reflected component I1 and the internally-scattered component I2 are reflected or scattered to change their traveling directions, and partially arrive at the photodetector 30.

First, a method for acquiring the internally-scattered component I2 is described. The light source 20 repeatedly emits a light pulse more than once at predetermined time intervals or predetermined timings in accordance with an instruction from the control circuit 60. A light pulse that is emitted from the light source 20 may for example be a rectangular wave whose fall time is close to zero. The term “fall time” herein means a period of time from the start to the end of a decrease in intensity of the light pulse. In general, light having entered the user 10 propagates through various paths within the user 10 and exits the user 10 through the surface with a time lag. For this reason, the rear end of the internally-scattered component I2 of a light pulse has a spread. In a case where the target part 10t of the user 10 is the forehead, the spread of the rear end of the internally-scattered component I2 is approximately 4 ns. With this in consideration, the fall time of the light pulse may be set, for example, to 2 ns or shorter, which is shorter than or equal to a half of 4 ns. The fall time may be 1 ns or shorter, which is shorter than or equal to a half of 2 ns. The rise time of a light pulse that is emitted from the light source 20 is arbitrary. The term “rise time” herein means a period of time from the start to the end of an increase in intensity of the light pulse. In detection of the internally-scattered component I2 according to the present embodiment, the falling portion of a light pulse is used, and the rising portion is not used. The rising portion of a light pulse may be used in detection of the surface-reflected component I1. The light source 20 may for example be a laser such as an LD. Light that is emitted from the laser has such a steep time response characteristic that the falling portion of a light pulse is substantially orthogonal to a time axis.

The wavelength of light that is emitted from the light source 20 may be any wavelength falling within a wavelength range of, for example, longer than or equal to 650 nm to shorter than or equal to 950 nm. This wavelength range falls within a wavelength range of red to near-infrared rays. The term “light” is herein used not only for visible light but also for infrared rays. The aforementioned wavelength range, called “biological window”, has the property of being comparatively hardly absorbed into moisture in a living organism or the skin. In a case where a living organism is a target of detection, using light falling within the aforementioned wavelength range makes it possible to achieve high detection sensitivity. As in the case of the present embodiment, in a case where blood flow changes in the skin and brain of the user 10 are detected, the light used is conceivably absorbed primarily into oxyhemoglobin (HbO₂) or deoxyhemoglobin (Hb). Oxyhemoglobin and deoxyhemoglobin differ in wavelength dependency of light absorption from each other. In general, a change in blood flow leads to changes in concentration of oxyhemoglobin and deoxyhemoglobin. These changes entail a change in degree of absorption of light. Accordingly, a change in blood flow leads to a temporal change in the amount of light that is detected.

The light source 20 may emit two or more wavelengths of light falling within the aforementioned wavelength range. Such a plurality of wavelengths of light may be emitted from a plurality of light sources, respectively. When two different wavelengths of light are emitted from two light sources, respectively, the optical path lengths of two wavelengths of light returning to the photodetector 30 via the target part 10t of the user 10 are designed to be substantially equal to each other. In this design, for example, the distance between the photodetector 30 and one of the light sources and the distance between the photodetector 30 and the other one of the light sources match, and the two light sources are placed in such locations as to be rotationally symmetrical with each other about the photodetector 30.

In order to make a measurement of the user 10 in a noncontact manner, the optical measuring device 100 according to the present embodiment may use a light source 20 designed in consideration of the impact on the retina. For example, a light source 20 that satisfies Class 1 of laser safety standards formulated separately by each country may be used. In a case where Class 1 is satisfied, the user 10 is irradiated with such low-intensity light that an accessible emission limit (AEL) falls below 1 mW. It should be noted that the light source 20 per se does not need to satisfy Class 1. For example, Class 1 of the laser safety standards may be satisfied by diffusing or attenuating light by placing a diffusing plate or an ND filter in front of the light source 20.

Conventionally, a streak camera has been used to distinctively detect information such as absorption coefficients and scattering coefficients at different in-depth locations within a living organism. For example, Japanese Unexamined Patent Application Publication No. 4-189349 discloses an example of such a streak camera. In these streak cameras, ultrashort light pulses femtoseconds or picoseconds in pulse duration have been used to make measurements with desired spatial resolution.

On the other hand, the optical measuring device 100 according to the present embodiment can detect the surface-reflected component I1 and the internally-scattered component I2 in distinction from each other. Accordingly, a light pulse that is emitted from the light source 20 does not need to be an ultrashort light pulse, and the pulse duration of a light pulse that is emitted from the light source 20 can be arbitrarily selected.

In a case where a cerebral blood flow measurement is made by irradiating the head of the user 10 with light, the amount of light of the internally-scattered component I2 may assume a very small value of approximately several thousandths to several ten thousandths of the amount of light of the surface-reflected component I1. Furthermore, with the laser safety standards in consideration, the amount of light with which the head of the user 20 can be irradiated is extremely small. This makes it very difficult to detect the internally-scattered component I2. Even in that case, if the light source 20 emits a light pulse whose pulse duration is comparatively long, the amount of integration of the internally-scattered component I2 with a time delay can be increased. This makes it possible to increase the amount of light detected and bring about improvement in SN ratio.

The light source 20 emits a light pulse whose pulse duration is for example longer than or equal to 3 ns. In general, light scattered within biological tissue such as a brain has a temporal spread of approximately 4 ns. FIG. 1B is a diagram showing examples of time variations in the intensity of light that arrives at the photodetector 30. FIG. 1B shows examples of three cases where the duration of an input light pulse that is emitted from the light source 20 is 0 ns, where the duration of an input light pulse that is emitted from the light source 20 is 3 ns, and where the duration of an input light pulse that is emitted from the light source 20 is 10 ns. As shown in FIG. 1B, an increase in duration of a light pulse from the light source 20 leads to an increase in amount of light of an internally-scattered component I2 that appears at the rear-end portion of a light pulse returning from the user 10.

FIG. 1C is a diagram whose horizontal axis represents the duration of an input light pulse and whose vertical axis represents the amount of light detected by the photodetector 30. The photodetector 30 includes an electronic shutter. FIG. 1C shows a result obtained under conditions where the electronic shutter was opened after a period of 1 ns has elapsed since the point of time at which the rear end of the light pulse had arrived at the photodetector 30 after having been reflected off the surface of the user 10. These conditions were selected because the surface-reflected component I1 is higher in proportion than the internally-scattered component I2 immediately after arrival of the rear end of the light pulse. As shown in FIG. 1C, when the pulse duration of a light pulse that is emitted from the light source 20 is longer than or equal to 3 ns, the amount of light detected can be maximized.

The light source 20 may emit a light pulse whose pulse duration is longer than or equal to 5 ns or, furthermore, longer than or equal to 10 ns. Meanwhile, too long pulse duration results in a wasteful increase in light that is not used. For this reason, the light source 20 emits a light pulse whose pulse duration is for example narrower than or equal to 50 ns. Alternatively, the light source 20 may emit a light pulse whose pulse duration is narrower than or equal to 30 ns or, furthermore, narrower than or equal to 20 ns.

A irradiation pattern of the light source 20 may be a pattern having a uniform intensity distribution within an irradiated region. In this respect, the present embodiment differs, for example, from a conventional optical measuring apparatus disclosed in Japanese Unexamined Patent Application Publication No. 11-164826. In the device disclosed in Japanese Unexamined Patent Application Publication No. 11-164826, a detector and a light source are approximately 3 cm away from each other, and a surface-reflected component is spatially separated from an internally-scattered component. This inevitably results in discrete photoirradiation. On the other hand, the optical measuring device 100 according to the present embodiment can reduce the surface-reflected component I1 by temporally separating it from the internally-scattered component I2. This makes it possible to use a light source 20 whose irradiation pattern has a uniform intensity distribution. An irradiation pattern having a uniform intensity distribution may be formed by diffusing, with a diffusing plate, light that is emitted from the light source 20.

Unlike the conventional technology, the present embodiment can detect the internally-scattered component I2 even directly beneath a point of irradiation of the user 10. The present embodiment can also enhance measuring resolution by irradiating the user 10 with light over a spatially wide range.

### 1-2. Photodetector 30

The photodetector 30 outputs a signal representing the amount of light of at least a portion of light emitted from the light source 20 and returning from the target part 10t of the user 10. This signal is for example a signal corresponding to the intensity of a reflected light pulse included in at least a portion of the rise time or a signal corresponding to the intensity of a reflected light pulse included in at least a portion of the fall time.

The photodetector 30 may include a plurality of photoelectric conversion elements 32 and a plurality of charge accumulators 34. Specifically, the photodetector 30 may include a plurality of photodetection cells arranged two-dimensionally. Such a photodetector 30 may acquire two-dimensional information on the user 10 at once. The photodetection cells are herein also referred to as “pixels”. The photodetector 30 may for example be any imaging element such as a CCD image sensor or a CMOS image sensor. More generally, the photodetector 30 includes at least one photoelectric conversion element 32 and at least one charge accumulator 34.

The photodetector 30 may include an electronic shutter. The electronic shutter is a circuit that controls a timing of imaging. In the present embodiment, the detector controller 62 of the control circuit 60 functions as the electronic shutter. The electronic shutter controls the duration of a single signal accumulation by which received light is converted into an effective electric signal to be accumulated and the duration of a stoppage of signal accumulation. A signal accumulation period can also be referred to as “exposure period”. In the following description, the duration of an exposure period is sometimes referred to as “shutter duration”. A period of time from the end of a single exposure period to the start of the next exposure period is sometimes referred to as “non-exposure period”. In the following, “OPEN” refers to a state where an exposure is being made, and “CLOSE” refers to a state where an exposure is under suspension.

The electronic shutter allows the photodetector 30 to adjust an exposure period and a non-exposure period within a range of subnano-seconds, e.g. from 30 ps to 1 ns. A conventional TOF camera intended to measure distance detects all of light emitted from the light source 20, reflected off a subject, and returning from the subject. The shutter duration of the conventional TOF camera needs to be longer than the pulse duration of light. On the other hand, the optical measuring device 100 according to the present embodiment does not need to correct the amount of light of a subject. For this reason, the shutter duration does not need to be longer than the pulse duration. The shutter duration can be set, for example, to a value that is greater than or equal to 1 ns and less than or equal to 30 ns. The optical measuring device 100 according to the present embodiment, whose shutter duration can be reduced, makes it possible to reduce the influence of a dark current contained in a detection signal.

In a case where information such as cerebral blood flow is detected by irradiating the head of the user 10 with light, the light is internally attenuated at a very high rate. For example, emitted light may be attenuated to approximately a millionth of incident light. For this reason, irradiation with one pulse alone may be insufficient in amount of light to detect the internally-scattered component I2. Irradiation in Class 1 of the laser safety standards is weak especially in amount of light. In this case, the light source 20 emits a light pulse more than once, and the photodetector 30 makes an exposure more than once accordingly, whereby improvement in sensitivity can be brought about by integrating detection signals.

The following describes an example configuration of the photodetector 30.

The photodetector 30 may include a plurality of pixels arrayed two-dimensionally on an imaging surface. Each pixel may include a photoelectric conversion element such as a photodiode and one or more charge accumulators. The following describes an example in which each pixel includes a photoelectric conversion element that generates, through a photoelectric conversion, signal charge corresponding to the amount of light received, a charge accumulator that accumulates signal charge generated by the surface-reflected component I1 of a light pulse, and a charge accumulator that accumulates signal charge generated by the internally-scattered component I2 of the light pulse. In the following example, the control circuit 60 causes the photodetector 30 to detect the surface-reflected component I1 by detecting a portion of a light pulse returning from the head of the user 10 that precedes the start of a falling edge. The control circuit 60 also causes the photodetector 30 to detect the internally-scattered component I2 by detecting a portion of the light pulse returning from the head of the user 10 that follows the start of the falling edge. In this example, the light source 20 emits two types of wavelength of light.

FIG. 1D is a diagram schematically showing an example configuration of one pixel 201 of the photodetector 30. It should be noted that FIG. 1D schematically shows a configuration of one pixel 201 and does not necessarily reflect the actual structure. In this example, the pixel 201 includes a photodiode 203, which performs photoelectric conversion; a first floating diffusion (FD) layer 204, a second floating diffusion layer 205, a third float diffusion layer 206, and a fourth float diffusion layer 207, which serve as charge accumulators; and a drain 202, which emits signal charge.

A photon having fallen on each pixel due to the emission of a single light pulse is converted by the photodiode 203 into a signal electron that serves as signal charge. The signal electron thus obtained is emitted to the drain 202 or sorted into any of the first to fourth floating diffusion layers 204 to 207 in accordance with a control signal that is inputted from the control circuit 60.

The emission of a light pulse from the light source 20, the accumulation of signal charge in the first floating diffusion layer 204, the second floating diffusion layer 205, the third float diffusion layer 206, and the fourth float diffusion layer 207, and the emission of signal charge to the drain 202 are repeatedly performed in this order. This repetitive operation is performed at a high speed and, for example, may be repeated several tens of thousands of times to several hundreds of millions of times within the duration of one frame of a moving image. The duration of one frame is for example approximately 1/30 second. The pixel 201 finally generates and output four image signals based on signal charge accumulated in the first to fourth floating diffusion layers 204 to 207.

In this example, the control circuit 60 causes the light source 20 to repeatedly emit a first light pulse and a second light pulse in sequence. The first light pulse has a first wavelength, and the second light pulse has a second wavelength. By selecting, as the first wavelength and the second wavelength, two wavelengths differing in absorptance from each other in internal tissue of the user 10, a state of the user 10 can be analyzed. For example, a wavelength that is longer than 805 nm may be selected as the first wavelength, and a wavelength that is shorter than 805 nm may be selected as the second wavelength. This makes it possible to detect changes in concentration of oxyhemoglobin and deoxyhemoglobin in blood of the user 10.

The control circuit 60 first causes the light source 20 to emit the first light pulse. The control circuit 60 causes the first floating diffusion layer 204 to accumulate signal charge in a first period during which the surface-reflected component I1 of the first light pulse is falling on the photodiode 203. Then, the control circuit 60 causes the second floating diffusion layer 205 to accumulate signal charge in a second period during which the internally-scattered component I2 of the first light pulse is falling on the photodiode 203. Next, the control circuit 60 causes the light source 20 to emit the second light pulse. The control circuit 60 causes the third floating diffusion layer 206 to accumulate signal charge in a third period during which the surface-reflected component I1 of the second light pulse is falling on the photodiode 203. Then, the control circuit 60 causes the fourth floating diffusion layer 207 to accumulate signal charge in a fourth period during which the internally-scattered component I2 of the second light pulse is falling on the photodiode 203.

In this way, the control circuit 60 causes the first floating diffusion layer 204 and the second diffusion layer 205 to sequentially accumulate signal charge from the photodiode 203 with a predetermined time lag after the start of the emission of the first light pulse. After that, the control circuit 60 causes the third floating diffusion layer 206 and the fourth diffusion layer 207 to sequentially accumulate signal charge from the photodiode 203 with a predetermined time lag after the start of the emission of the second light pulse. These operations are repeated more than once. For estimation of the amounts of disturbance light and ambient light, a period may be provided during which signal charge is accumulated in other floating diffusion layers (not illustrated) with the light source 20 turned off. By subtracting the amount of signal charge in the other floating diffusion layers from the amount of signal charge in the first to fourth floating diffusion layers 204 to 207, a signal from which disturbance light and ambient light components have been eliminated may be obtained.

It should be noted that the present embodiment, in which the number of charge accumulators is 4, may alternatively be designed for any purpose so that the number of charge accumulator is larger than or equal to 2. For example, in a case where only one type of wavelength is used, the number of charge accumulators may be 2. Alternatively, in an application when one type of wavelength is used and the surface-reflected component I1 is not detected, the number of charge accumulator for each pixel may be 1. Alternatively, even in a case where two or more types of wavelength are used, the number of charge accumulators may be 1, provided imaging is performed in a separate frame using each wavelength. Alternatively, as will be mentioned later, the number of charge accumulators may be 1, provided detection of the surface-reflected component I1 and detection of the internally-scattered component I2 are performed in separate frames, respectively.

FIG. 1E is a diagram showing an example configuration of the photodetector 30. In FIG. 1E, a region surrounded by a frame of chain double-dashed lines is equivalent to one pixel 201. The pixel 201 includes one photodiode. Although FIG. 1E shows only four pixels arrayed in two rows and two columns, a larger number of pixels may be arranged in actuality. The pixel 201 includes first to fourth floating diffusion layers 204 to 207. Signals that are accumulated in the first to fourth floating diffusion layers 204 to 207 are treated as if they are signals of four pixels of a common CMOS image sensor, and are outputted from the photodetector 30.

Each pixel 201 has four signal detection circuits. Each signal detection circuit includes a source follower transistor 309, a row selection transistor 308, and a reset transistor 310. In this example, the reset transistor 310 corresponds to the drain 202 shown in FIG. 1D, and a pulse that is inputted to a gate of the reset transistor 310 corresponds to a drain-emitted pulse. Each transistor is for example, but is not limited to, a field-effect transistor formed on a semiconductor substrate. As illustrated, one of an input terminal and an output terminal of the source follower transistor 309 and one of an input terminal and an output terminal of the row selection transistor 308 are connected to each other. The one of the input terminal and the output terminal of the source follower transistor 309 is typically a source. The one of the input terminal and the output terminal of the row selection transistor 308 is typically a drain. A gate, i.e. a control terminal, of the source follower transistor 309 is connected to the photodiode 203. Signal charge of a hole or electron generated by the photodiode 203 is accumulated in a floating diffusion layer, i.e. a charge accumulator, between the photodiode 203 and the source follower transistor 309.

Although not shown in FIG. 1E, the first to fourth floating diffusion layers 204 to 207 are connected to the photodiode 203. A switch may be provided between the photodiode 203 and each of the first to fourth floating diffusion layers 204 to 207. This switch switches between states of conduction between the photodiode 203 and a corresponding one of the first to fourth floating diffusion layers 204 to 207 in accordance with a signal accumulation pulse from the control circuit 60. This controls the start and stoppage of accumulation of signal charge in each of the first to fourth floating diffusion layers 204 to 207. The electronic shutter according to the present embodiment has a mechanism for such exposure control.

Signal charge accumulated in the first to fourth floating diffusion layers 204 to 207 is read out by a gate of the row selection transistor 308 being turned on by a row selection circuit 302. In so doing, a current that flows from a source follower power source 305 to the source follower transistor 309 and a source follower load 306 is amplified according to signal potentials of the first to fourth floating diffusion layers 204 to 207. An analog signal derived from this current that is read out from a vertical signal line 304 is converted into digital signal data by an analog-digital (AD) converter circuit 307 connected for each column. This digital signal data is read out for each column by a column selection circuit 303, and is outputted from the photodetector 30. The row selection circuit 302 and the column selection circuit 303 read out one row first and then read out the next row. After that, the row selection circuit 302 and the column selection circuit 303 similarly read out information on signal charge in floating diffusion layers in all rows. The control circuit 60 resets all floating diffusion layers by turning on the gate of the reset transistor 310 after having read out all signal charge. With this, one frame of imaging is completed. After that, frames of imaging are similarly repeated at a high speed, whereby a series of frames of imaging by the photodetector 30 is completed.

In the present embodiment, an example of a CMOS photodetector 30 is described. Alternatively, the photodetector 30 may be a different type of imaging element. The photodetector 30 may for example be a CCD, a single-photon digital element, or an amplifier image sensor such as an EMCCD or an ICCD.

FIG. 1F is a diagram showing an example of operation within one frame. As shown in FIG. 1F, the emission of the first light pulse and the emission of the second light pulse may be alternately switched between more than once within one frame. This makes it possible to reduce a time lag between timings of acquisition of detection images by the two types of wavelength and, even when the user 10 is moving, take photographs with the first light pulse and the second light pulse almost at the same time.

In the present embodiment, the photodetector 30 can detect the surface-reflected component I1 and/or the internally-scattered component I2 of a light pulse. First biological information on the user 10 can be acquired from a temporal or spatial change in the surface-reflected component I1. The first biological information may for example be the pulse of the user 10. Meanwhile, brain activity information, i.e. second biological information on the user 10, can be acquired from a temporal or spatial change in the internally-scattered component I2.

The first biological information may be acquired by a method that is different from a method for detecting the surface-reflected component I1. For example, the first biological information may be acquired by utilizing a different type of detector that is different from the photodetector 30. In that case, the photodetector 30 detects only the internally-scattered component I2. The different type of detector may for example be a radar or a thermography. The first biological information may for example be at least one selected from the group consisting of the pulse, perspiration, respiration, and body temperature of the user 10. The first biological information is biological information other than brain activity information that is obtained by detecting the internally-scattered component I2 of a light pulse with which the head of the user 10 was irradiated. The phrase “other than brain activity information” here is not intended to mean that the first biological information contains no information attributed to brain activity. The first biological information contain biological information attributed to biological activity that is different from brain activity. The first biological information may for example be biological information attributed to autonomous or reflex biological activity.

### 1-3. Control Circuit 60 and Signal Processing Circuit 70

The control circuit 60 adjusts a time lag between an emission timing of a light pulse from the light source 20 and a shutter timing of the photodetector 30. The time lag is herein sometimes referred to as “phase difference”. The “emission timing” of the light source 20 is a timing at which a light pulse that is emitted from the light source starts to rise. The “shutter timing” is a timing at which an exposure is started. The control circuit 60 may adjust the phase difference by changing the emission timing or may adjust the phase difference by changing the shutter timing.

The control circuit 60 may be configured to eliminate an offset component from a signal detected by each pixel of the photodetector 30. The offset component is a signal component derived from ambient light such as sunlight or fluorescent light or disturbance light. An offset component derived from ambient light or disturbance light is estimated by detecting a signal with the photodetector 30 in a state where the light source 20 is de-actuated and no light is emitted from the light source 20.

The control circuit 60 may for example be a combination of a processor and a memory or an integrated circuit such as a microcontroller containing a processor and a memory. The control circuit 60 adjusts, for example, the emission timing and the shutter timing, for example, through the execution by the processor of a program recorded in the memory.

The signal processing circuit 70 is a circuit that processes an image signal outputted from the photodetector 30. The signal processing circuit 70 performs arithmetic processing such as image processing. The signal processing circuit 70 may be implemented, for example, as a combination of a digital signal processor (DSP), a programmable logic device (PLD) such as a field-programmable gate array (FPGA), a central processing unit (CPU), or a graphics processing unit (GPU) and a computer program. The control circuit 60 and the signal processing circuit 70 may be one integrated circuit or may be separated individual circuits. The signal processing circuit 70 may for example be a constituent element of an external device, such as a server, provided in a remote place. In this case, the external device, such as a server, mutually transmits and receives data to and from the light source 20, the photodetector 30, and the control circuit 60 either by radio or by wire.

The signal processing circuit 70 according to the present embodiment can generate, on the basis of a signal outputted from the photodetector 30, moving image data representing time variations in blood flow in the skin surface and cerebral blood flow. The signal processing circuit 70 may generate other information as well as such moving image data. For example, the signal processing circuit 70 may generate biological information such as a blood flow rate, blood pressure, a blood oxygen saturation level, or a heart rate in the brain by being synchronized with another device. The signal processing circuit 70 may estimate an offset component derived from disturbance light and eliminate such an offset component.

It is known that there is a close relationship between a change in cerebral blood flow rate or in a component in blood such as hemoglobin and human neural activity. For example, a change in activity of a neural cell according to a change in human emotion leads to a change in cerebral blood flow or in a component in blood. Accordingly, being able to measure biological information such as a change in cerebral blood flow or in a component in blood makes it possible to estimate a psychological state of the user 10. The psychological state of the user 10 means, for example, a feeling, an emotion, a state of health, or a temperature sense. The feeling may include a feeling such as pleasantness or unpleasantness. The emotion may include an emotion such as security, anxiety, sorrow, or resentment. The state of health may include a state such as vigor or fatigue. The temperature sense may include a sense such as hot, cold, or muggy. Derived from these, indices representing degrees of brain activity, such as a level of skill, a level of mastery, and a degree of concentration, may be included in the psychological state. The signal processing circuit 70 may estimate the psychological state, such as the degree of concentration, of the user 10, for example, on the basis of a change in cerebral blood flow rate and output a signal representing an estimation result.

FIG. 1G is a flow chart schematically showing an operation carried out by the control circuit 60 regarding the light source 20 and the photodetector 30. The control circuit 60 executes the operation schematically shown in FIG. 1G. In the operation described here, only the internally-scattered component I2 is detected.

In step S101, first, the control circuit 60 causes the light source 20 to emit a light pulse for a predetermined period of time. At this point in time, the electronic shutter of the photodetector 30 is suspending an exposure. The control circuit 60 causes the electronic shutter to suspend an exposure until the end of a period of time during which a portion of the light pulse is reflected off the surface of the user 10 and arrives at the photodetector 30. Next, in step S102, the control circuit 60 causes the electronic shutter to start an exposure at a timing when another portion of the light pulse is scattered inside the user 10 and arrives at the photodetector 30. After a predetermined period of period has elapsed, the control circuit 60 proceeds to step S103, in which the control circuit 60 causes the electronic shutter to suspend the exposure. Then, in step S104, the control circuit 60 determines whether the number of times the aforementioned signal accumulation was executed has reached a predetermined number of times. In a case where the control circuit 60 has determined in step S104 that the predetermined number of times has not been reached (No in step S104), steps S101 to S103 are repeated until the control circuit 60 determines in step S104 that the predetermined number of times has been reached (Yes in step S104). In a case where the control circuit 60 has determined in step S104 that the predetermined number of times has been reached (Yes in step S104), the control circuit 60 proceeds to step S105, in which the control circuit 60 causes the photodetector 30 to generate and output a signal representing an image based on signal charge accumulated in each floating diffusion layer.

The foregoing operation makes it possible to detect, with high sensitivity, a component of light scattered inside a target of measurement. It should be noted that it is not essential to emit light or make an exposure more than once, but it is only necessary to emit light or make an exposure on an as-needed basis.

### 1-4. Other

The optical measuring device 100 may include an image-forming optical system that forms a two-dimensional image of the user 10 on a photoreceptive surface of the photodetector 30. The image-forming optical system has an optical axis that is substantially orthogonal to the photoreceptive surface of the photodetector 30. The image-forming optical system may include a zoom lens. A change in position of the zoom lens leads to a change in magnification of the two-dimensional image of the user 10, so that the resolution of the two-dimensional image on the photodetector 30 changes. Accordingly, even at a long distance from the user 10, a desired measurement region can be enlarged for detailed observation.

The optical measuring device 100 may include a bandpass filter, provided between the user 10 and the photodetector 30, that allows passage of only a band of wavelength of light that is emitted from the light source 20 or light therearound. This makes it possible to reduce the influence of a disturbance component such as ambient light. The bandpass filter may be constituted, for example, by a multilayer filter or an absorption filter. In consideration of a change in temperature of the light source 20 and a shift in band due to oblique incidence on the filter, the bandwidth of the bandpass filter may range from approximately 20 to 100 nm.

The optical measuring device 100 may include polarizing plates between the light source 20 and the user 10 and between the photodetector 30 and the user 10, respectively. In this case, the direction of polarization of the polarizing plate disposed beside the light source 20 and the direction of polarization of the polarizing plate disposed beside the photodetector 30 may be in an orthogonal nicole relationship with each other. This makes it possible to prevent a specularly-reflected component of the surface-reflected component I1 of the user 10, i.e. a component whose angle of incidence and angle of reflection are equal, from arriving at the photodetector 30. That is, this makes it possible to reduce the amount of light in which the surface-reflected component I1 arrives at the photodetector 30.

### 2. Operation of Light Source and Photodetector

The optical measuring device 100 according to the present embodiment can detect the surface-reflected component I1 and the internally-scattered component I2 in distinction from each other. In a case where the target part 10t of the user 10 is the forehead, signal strength based on the internally-scattered component I2 that needs to be detected is very low. This is because, as mentioned above, the scattering and absorption of light by the scalp, cerebrospinal fluid, the skull, gray matter, white matter, and blood flow are great in addition to irradiation with light of a very low light level that satisfies the laser safety standards. Furthermore, a change in signal strength due to a change in blood flow rate or in a component in the bloodstream during brain activity is so small as to be equivalent to several tenths of a size. Accordingly, in a case where the internally-scattered component I2 is detected, the surface-reflected component I1, which is several thousand times to several tens of thousands of times the signal component that needs to be detected, is eliminated as much as possible during imaging.

The following describes an example of operation of the light source 20 and the photodetector 30 in the optical measuring device 100 detecting the internally-scattered component I2.

As shown in FIG. 1A, irradiation of the target part 10t of the user 10 with a light pulse from the light source 20 generates a surface-reflected component I1 and an internally-scattered component I2. The surface-reflected component I1 and the internally scattered component I2 partially arrive at the photodetector 30. The internally-scattered component I2 passes through the interior of the user 10 for a period from emission from the light source 20 to arrival at the photodetector 30. For this reason, the internally-scattered component I2 is longer in optical path length than the surface-reflected component I1. Accordingly, the internally-scattered component I2 arrives at the photodetector 30 at a later time on average than the surface-reflected component I1 arrives at the photodetector 30.

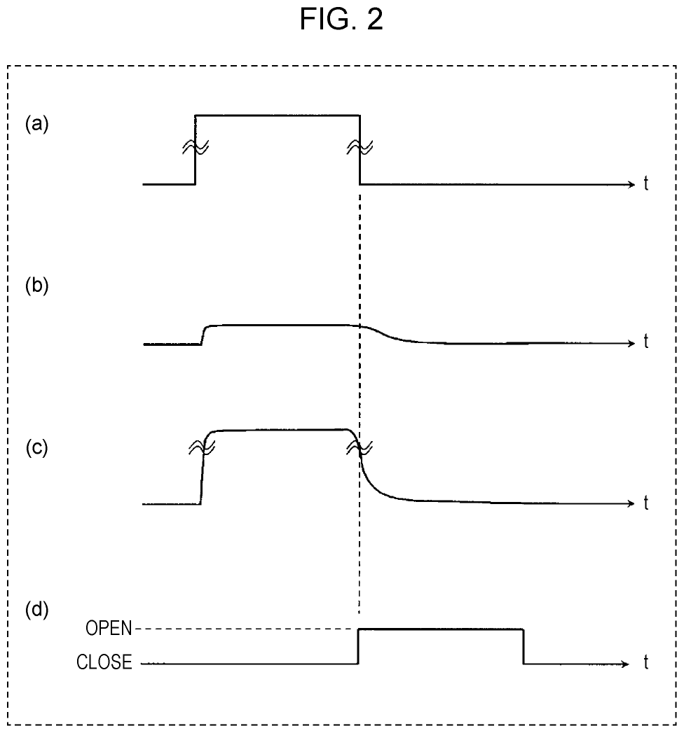

FIG. 2 is a diagram representing optical signals generated by the emission of rectangular light pulses from the light source 20 and the arrival at the photodetector 30 of light returning from the user 10. The horizontal axis represents time (t) for the signals (a) to (d) of FIG. 2. The vertical axis represents strength for the signals (a) to (c) of FIG. 2, and represents an OPEN or CLOSE state of the electronic shutter for the signal (d) of FIG. 2. The signal (a) of FIG. 2 indicates a surface-reflected component I1. The signal (b) of FIG. 2 indicates an internally-scattered component I2. The signal (c) of FIG. 2 indicates a combined component of the surface-reflected component I1 indicated by the signal (a) of FIG. 2 and the internally-scattered component I2 indicated by the signal (b) of FIG. 2. As indicated by the signal (a) of FIG. 2, the surface-reflected component I1 maintains an almost rectangular waveform. Meanwhile, the internally-scattered component I2 is a combination of lights of various optical path lengths. For this reason, as indicated by (b) of FIG. 2, the internally-scattered component I2 exhibits such a characteristic that the rear end of the light pulse leaves a long, thin trail. In other words, the fall time of the internally-scattered component I2 is longer than the fall time of the surface-reflected component I1. As indicated by the signal (d) of FIG. 2, for extraction of the internally-scattered component I2 at a high rate from the optical signal indicated by the signal (c) of FIG. 2, the electronic shutter starts an exposure at or after a time point at which the rear end of the surface-reflected component I1 arrives. In other words, an exposure is started when or after the waveform of the surface-reflected component I1 has fallen. This shutter timing is adjusted by the control circuit 60.

In a case where a physical object of measurement is not planar, the pixels of the photodetector 30 vary in timing of arrival of light from one another. In this case, the shutter timing indicated by the signal (d) of FIG. 2 may be individually determined for each pixel. For example, assume that a direction perpendicular to the photoreceptive surface of the photodetector 30 is a z direction. The control circuit 60 acquires, in advance, data representing a z-coordinate two-dimensional distribution on the surface of the target part and vary the shutter timing for each pixel on the basis of this data. This makes it possible to, even in a case where the surface of the target part is curved, determine an optimum shutter timing for each location.

In the example indicated by the signal (a) of FIG. 2, the rear end of the surface-reflected component I1 vertically falls. In other words, there is no amount of time from the start to the end of the falling edge of the surface-reflected component I1. However, in reality, there is a case where the rear end of the surface-reflected component I1 does not vertically fall. For example, in a case where the falling edge of the waveform of a light pulse that is emitted from the light source 20 is not completely vertical, a case where there are fine asperities on the surface of the target part, or a case where there occurs scattering in the epidermis, the rear end of the surface-reflected component I1 does not vertically fall. Further, since the user 10 is an opaque object, the surface-reflected component I1 is much larger in amount of light than the internally-scattered component I2. Accordingly, even in a case where the rear end of the surface-reflected component I1 slightly strays from the time point of vertical falling, the internally-scattered component I2 might be buried. Furthermore, there is also a case where a time delay occurs along with electron transfer during a period of readout by the electronic shutter. For these reasons, ideal binary readout may not be achieved as indicated by the signal (d) of FIG. 2. In that case, the control circuit 60 may make the timing of shutter start of the electronic shutter slightly later than a time immediately following the falling edge of the surface-reflected component I1. For example, the delay may range from approximately 0.5 ns to 5 ns. Instead of adjusting the shutter timing of the electronic shutter, the control circuit 60 may adjust the emission timing of the light source 20. In other words, the control circuit 60 may adjust a time lag between the shutter timing of the electronic shutter and the emission timing of the light source 20. In a case where a change in blood flow rate or in a component in the bloodstream in the brain is measured in a non-contact manner, making the shutter timing too late further decreases the internally-scattered component I2, which is inherently small. For this reason, the shutter timing may held near the rear end of the surface-reflected component I1. As mentioned above, the time delay due to scattering in the forehead is approximately 4 ns. In this case, the maximum amount of delay in the shutter timing may be approximately 4 ns.

Signals may be accumulated by making exposures at shutter timings at equal time intervals to each of the plurality of light pulses emitted from the light source 20. This amplifies the amount of light in which the internally-scattered component I2 is detected.

An offset component may be estimated by taking photographs in the same exposure period with no light emitted from the light source 20 instead of or in addition to placing the bandpass filter between the user 10 and the photodetector 30. The offset component thus estimated is eliminated by subtraction from a signal detected by each pixel of the photodetector 30. This makes it possible to eliminate a dark-current component that is generated on the photodetector 30.

The internally-scattered component I2 contains internal property information, e.g. cerebral blood flow information, on the user 10. The amount of light that is absorbed into blood changes according to temporal fluctuations in cerebral blood flow rate of the user 10. As a result, the amount of light that is detected by the photodetector 30 increases or decreases accordingly. This makes it possible to estimate a state of brain activity from a change in cerebral blood flow rate of the user 10 by monitoring the internally-scattered component I2.

Next, an example of a method for detecting the surface-reflected component I1 is described. The surface-reflected component I1 contains surface property information, e.g. facial and scalp blood flow information, on the user 10.

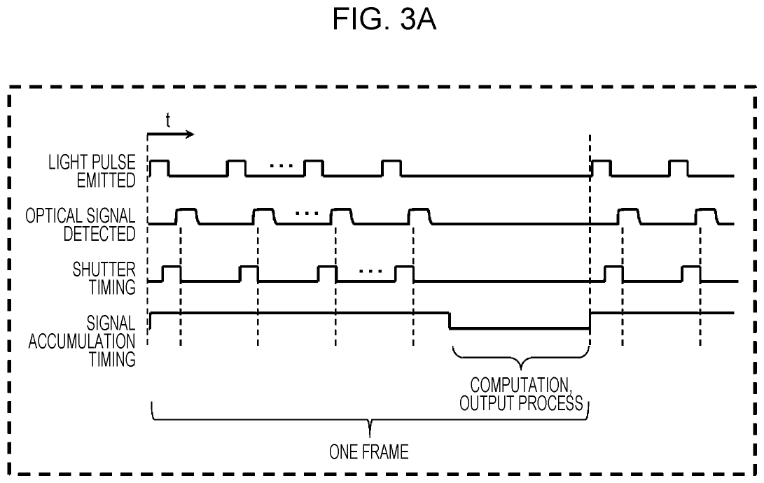

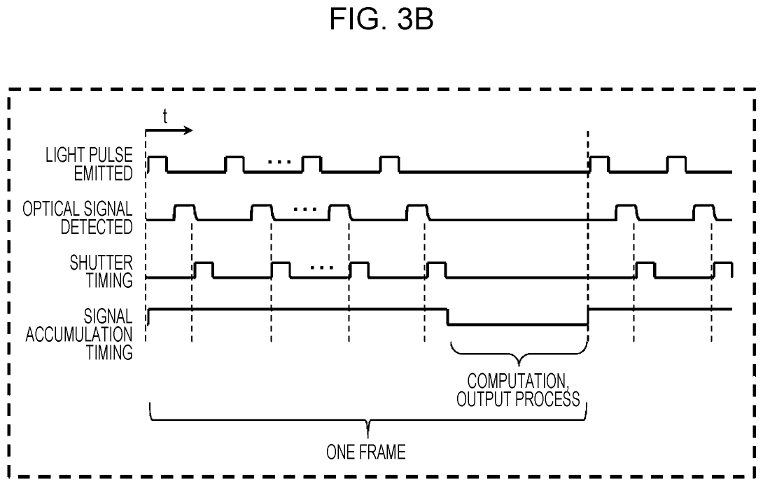

FIG. 3A is a diagram schematically showing an example of a timing chart in a case where the surface-reflected component I1 is detected. For example, as shown in FIG. 3A, for detection of the surface-reflected component I1, the shutter may be made OPEN before a light pulse arrives at the photodetector 30, and the shutter may be made CLOSE before the rear end of the light pulse arrives. Controlling the shutter in this way makes it possible to reduce mixing of the internally-scattered component I2. This in turn makes it possible to increase the proportion of light having passed near the surface of the user 10. The shutter CLOSE timing may immediately follow the arrival of light at the photodetector 30. This enables signal detection with a high proportion of the surface-reflected component I1, whose optical path length is comparatively short. Acquiring a signal of the surface-reflected component I1 makes it possible to detect the pulse or degree of oxygenation of facial blood flow or the user 10. As another method for acquiring the surface-reflected component I1, the photodetector 30 may detect the entirety of a light pulse or detect continuous light emitted from the light source 20.

FIG. 3B is a diagram schematically showing an example of a timing chart in a case where the internally-scattered component I2 is detected. A signal of the internally-scattered component I2 can be acquired by making the shutter OPEN during a period of time in which the rear-end portion of a pulse arrives at the photodetector 30.

In a summary of the operation shown in FIG. 3B, the control circuit 60 performs the following operation. The control circuit 60 causes the light source 20 to emit one or more light pulses. The control circuit 60 causes the photodetector 30 to detect a component included in the fall time of each light pulse returning from the target part 10t of the user 10. This component contains the internally-scattered component I2. The control circuit 60 causes the photodetector 30 to output a signal obtained by this detection. The signal processing circuit 70 generates, in accordance with this signal, a signal representing a state of brain activity of the user 10.

The surface-reflected component I1 may be detected by a device other than the optical measuring device 100, which acquires the internally-scattered component I2. A device separate from a device that acquires the internally-scattered component I2 or a separate device such as a sphygmometer or a Doppler blood flowmeter may be used. In that case, the separate device is used in consideration of timing synchronization between the devices, interference of light, and adjustment of places of detection. Performing time-division imaging with an identical camera or an identical sensor as in the case of the present embodiment makes it hard for temporal or spatial discrepancies to occur. In a case where signals of both the surface-reflected component I1 and the internally-scattered component I2 are acquired by an identical sensor, as described with reference to FIGS. 3A and 3B, it is possible to switch, frame by frame, between acquiring one component and acquiring another component. Alternatively, as described with reference to FIGS. 1D to 1F, it is possible to alternately switch, at a high speed within one frame, between acquiring one component and acquiring another component. This makes it possible to reduce a time lag between detection of the surface-reflected component I1 and detection of the internally-scattered component I2.

Furthermore, each of the signals of the surface-reflected component I1 and the internally-scattered component I2 may be acquired using two wavelengths of light. For example, light pulses of two wavelengths of 750 nm and 850 nm may be used. This makes it possible to calculate a change in concentration of oxyhemoglobin and a change in concentration of deoxyhemoglobin from changes in amount of light detected at each of the wavelengths. In a case where the surface-reflected component I1 and the internally-scattered component I2 are each acquired at two wavelengths, a method for switching among four types of charge accumulation at a high speed within one frame may be utilized, for example, as described with reference to FIGS. 1D to 1F. Such a method makes it possible to reduce a temporal discrepancy between detection signals.

A specific example of a method for acquiring biological information is described here.

A prominent role of blood is to receive oxygen from the lungs, carry oxygen to tissue, receive carbon dioxide from the tissue, and circulate carbon dioxide through the lungs. Approximately 15 g of hemoglobin is present in 100 ml of blood. Oxyhemoglobin is hemoglobin combined with oxygen, and deoxyhemoglobin is hemoglobin not combined with oxygen. As mentioned above, oxyhemoglobin and deoxyhemoglobin are different in optical absorbing characteristic from each other. Oxyhemoglobin relatively well absorbs near-infrared rays of a wavelength exceeding approximately 805 nm. On the other hand, deoxyhemoglobin relatively well absorbs near-infrared rays or red light of a wavelength shorter than 805 nm. Oxyhemoglobin and deoxyhemoglobin are about equal in absorptance with respect to near-infrared rays of a wavelength of 805 nm. Therefore, a first wavelength that is longer than 600 nm and shorter than 805 nm and a second wavelength that is longer than 805 nm and shorter than 1000 nm may be used. For example, the two wavelengths of light of 750 nm and 850 nm may be used. On the basis of these amounts of light detected, time variations in concentration of oxyhemoglobin and deoxyhemoglobin in blood can be detected. Furthermore, an oxygen saturation of hemoglobin can also be found. The oxygen saturation is a value that indicates what proportion of hemoglobin in blood is combined with oxygen. The oxygen saturation is defined by the following mathematical formula:

Oxygen saturation=C(HbO₂)/[C(HbO₂)+C(Hb)]×100(%),

where C(Hb) is the concentration of deoxyhemoglobin and C(HbO₂) is the concentration of oxyhemoglobin.

Although blood is not the only component contained in a living organism that absorbs red light and near-infrared light, a temporal fluctuation in light absorptance is attributed primarily to hemoglobin in the arterial blood. Therefore, a blood oxygen saturation level can be measured with high accuracy on the basis of a fluctuation in absorptance. Arterial blood ejected from the heart moves through blood vessels as a pulse wave. Meanwhile, venous blood has no pulse wave. Although light with which a living organism was irradiated passes through the living organism while being absorbed into each layer of the living organism such as tissue other than an artery, a vein, and blood, there is no temporal fluctuation in thickness of tissue other than an artery. For this reason, scattered light from within the living organism indicates a temporal change in intensity according to a change in thickness of an arterial blood layer due to pulsation. This change reflects the change in thickness of the arterial blood layer and does not include the influence of venous blood or tissue. Therefore, information on arterial blood can be obtained by focusing attention solely on a fluctuation component of the scattered light. The pulse can also be taken by measuring the cycle of a component that varies according to time.

The optical measuring device 100 can detect a change in amount of oxyhemoglobin of the scalp or face or the pulse from a temporal change in the surface-reflected component I1 by emitting a pulse of near-infrared light or visible light toward the head of the user 10. The light source 20 emits near-infrared light or visible light for acquisition of the surface-reflected component I1. In a case where near-infrared light is used, measurements can be made night and day. In a case where the pulse is measured, visible light may be used for higher sensitivity. During the daytime, solar radiation, which is disturbance light, or an indoor light source may be used instead of illumination. In the case of an insufficient amount of light, a dedicated light source may be used for reinforcement. The internally-scattered component I2 contains a light component having arrived at the brain. A temporal increase or decrease in cerebral blood flow can be measured by measuring a time variation in the internally-scattered component I2.

Light having arrived at the brain also passes through the scalp and the facial surface. For this reason, fluctuations in blood flow of the scalp and the face are also detected in a superimposed manner. In order to eliminate or reduce their influence, the signal processing circuit 70 may perform a process of subtracting the surface-reflected component I1 from the internally-scattered component I2 detected by the photodetector 30. This makes it possible to acquire pure cerebral blood flow information from which scalp and facial blood flow information has been excluded. The subtraction is done, for example, by using a method for subtracting, from a signal of the internally-scattered component I2, a value obtained by multiplying a signal of the surface-reflected component I1 by a coefficient of 1 or larger determined in consideration of a difference in optical path length. This coefficient may be calculated by a simulation or an experiment on the basis of an average value of optical constants of the heads of ordinary humans. Such a subtraction process can be easily performed in a case where measurements are made by an identical camera or sensor using an identical wavelength of light. This is because temporal and spatial discrepancies are easily reduced and a scalp blood flow component contained in the internally-scattered component I2 and a characteristic of the surface-reflected component I1 are easily matched with each other.

The skull exists between the brain and the scalp. For this reason, a two-dimensional distribution of cerebral blood flow and a two-dimensional distribution of blood flow of the scalp and the face are independent of each other. Accordingly, a two-dimensional distribution of the internally-scattered component I2 and a two-dimensional distribution of the surface-reflected component I1 may be separated from each other using a statistical method such as an independent component analysis or a principal component analysis on the basis of signals that are detected by the photodetector 30.

3. Measurement of Distance between Target Part of User and Photodetector

The following describes an example of a method for determining a shutter timing according to the distance between the target part 10t of the user 10 and the photodetector 30. In the following description, the distance between the center of the target part 10t of the user 10 and the center of the photoreceptive surface of the photodetector 30 is referred to as “measuring distance”.

The time it takes for light emitted from the light source 20 to return to the photodetector 30 depends on the distance the light traveled. Accordingly, the shutter timing may also be adjusted according to the measuring distance.

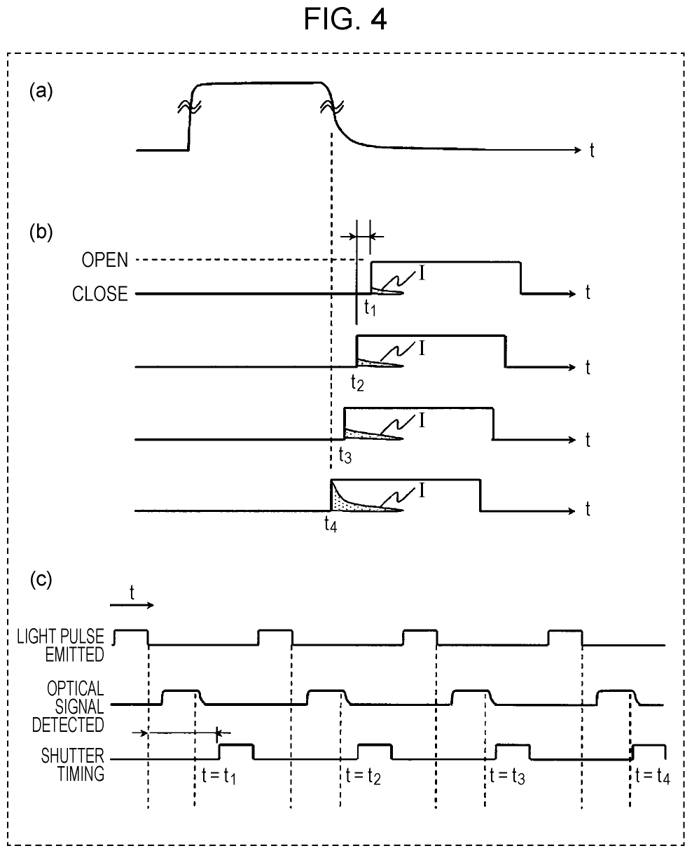

FIG. 4 is a diagram for explaining a method for determining an appropriate shutter timing according to the measuring distance. A portion (a) of FIG. 4 shows an example of a time response waveform of an optical signal that arrives at the photodetector 30. A portion (b) of FIG. 4 schematically shows a plurality of exposure periods differing in start point from one another and an example of the amount of light that is detected in each exposure period. In this example, first, the shutter is opened at a time point t=t₁ that is sufficiently later than the start of the falling edge of a reflected light pulse. At this point in time, an optical signal from the target part 10t is not contained in the image taken, or only a faint optical signal I at the end of a skirt is detected. This optical signal I is a signal that contains relatively much information on a deep place in the target part 10t, i.e. information on light whose optical path length is comparatively long. As for the next light pulse, the shutter is opened at a timing t=t₂ that is closer in shutter timing to the rear end of the pulse wave than t=t₁ by a particular period of time. Then, the amount of light in which the optical signal I is detected also changes according to the extent to which a shutter shift is made. Similarly, the control circuit 60 moves the shutter timing gradually closer to the rear end of the pulse wave by a particular period of time by adjusting the timing of start of an exposure to t=t₃ and then t=t₄. The timing at which the optical signal I started to sharply increase may be determined as a place that is equivalent to the starting point of the rear-end portion of the pulse wave. The particular period of time is a small value in comparison with the spread of the optical signal I at a skirt at the rear end of the pulse wave of the internally-scattered component I2. The particular period of time may be a value falling within a range of, for example, 30 ps to 1 ns.

A portion (c) of FIG. 4 shows a temporal relationship among an emitted light pulse from the light source 20, an optical signal on the photodetector 30, and the shutter timing. In this example, a light pulse is periodically emitted from the light source 20. After a previous shutter has closed, a next light pulse is emitted. Intervals between light pulses may be shorter than those illustrated. A shutoff period between two consecutive light pulses that are emitted from the light source 20 may for example be four or less times, twice or less times, or, furthermore, 1.5 or less times longer than the shutter duration. Alternatively, a shutoff period between two consecutive light pulses may be four or less times, twice or less times, or, furthermore, 1.5 or less times longer than the pulse duration. Such a shortening of intervals between consecutive light pulses makes it possible to increase the number of pulses that are integrated per unit time of photodetection by the photodetector 30. This in turn makes it possible to improve the sensitivity with which optical signals are acquired at a predetermined frame rate.

As a method for searching for an optimum shutter timing, a method other than the method shown in FIG. 4 for continuously varying the shutter timing may be used. For example, an iteration method such as a bisection method or a Newton's method or a numerical calculation method may be used. This makes it possible to reduce the number of shots and shorten search time.

The method shown in FIG. 4 does not involve a direct calculation of the distance to the target part 10t of the user 10. Instead of this method, for example, a measurement based on triangulation using a pantoscopic or binocular camera or a time-of-flight measurement using a TOF scheme may be used to directly measure the distance to determine the shutter timing.

An exposure to one light pulse alone is small in amount of light and may lead to deterioration of the SN ratio. In order to reduce deterioration of the SN ratio, it is possible to make an exposure more than once with identical time lags and integrate signals thus acquired.

Although the shutter timing is adjusted in the example shown in FIG. 4, the emission timing of the light source 20 may be adjusted instead of the shutter timing. In the case, the shutter timing may come at regular intervals.

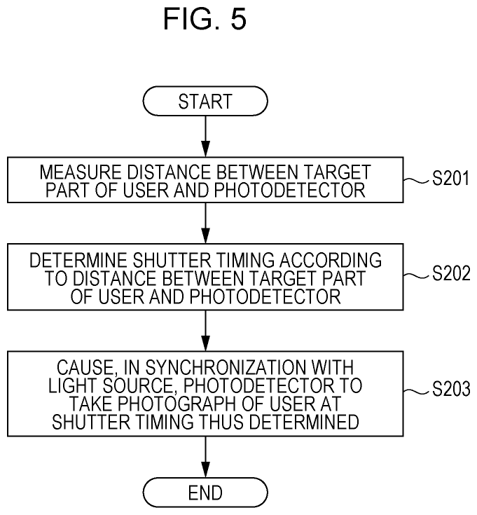

FIG. 5 is a flow chart showing an example of an operation of adjusting the shutter timing according to distance to a physical object. First, in step S201, the control circuit 60 conducts a measurement of the measuring distance. This measurement is not limited to the aforementioned method for directly measuring distance but may be a method for indirectly measuring distance. Next, in step S202, the control circuit 60 determines the shutter timing or the timing of an emitted light pulse according to the measuring distance. This timing may be set to a time when the shutter does not contain the surface-reflected component I1 of light returning from the target part 10t of the user 10. Furthermore, in step S203, the control circuit 60 causes, in synchronization with the light source 20, the photodetector 30 to take a photograph of the target part 10t of the user 10 at the shutter timing thus determined.

The pulse duration or shutter duration of the light source 20 that is used in an operation of determining the shutter timing or the emission timing of the light source 20 may be different from the pulse duration or shutter duration that is used in an operation of acquiring cerebral blood flow information on the user 10.

### 4. Example of Detection of Change in Cerebral Blood Flow Rate

The following describes an example of a method for detecting a change in cerebral blood flow rate of the user 10.

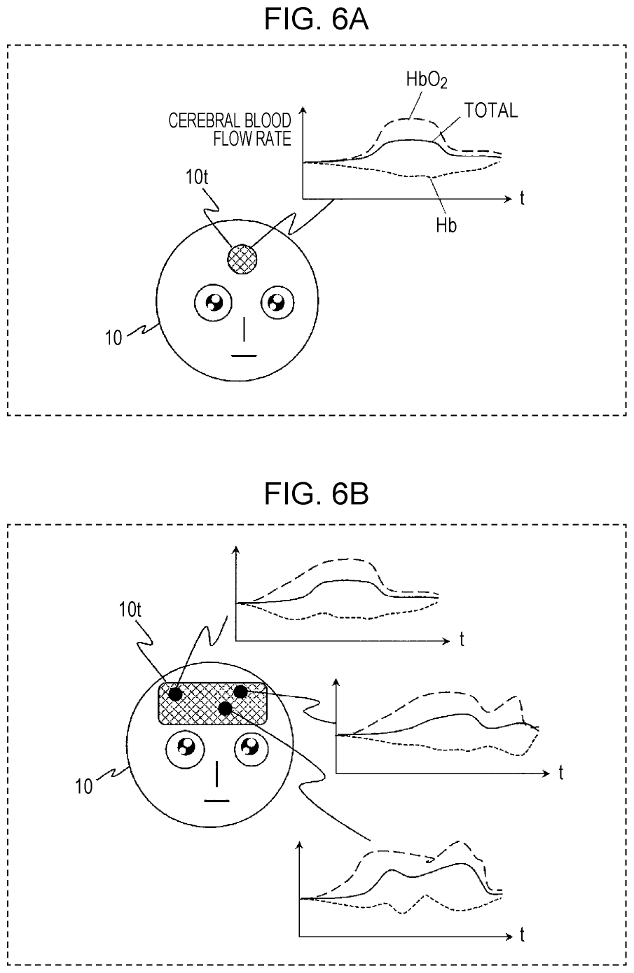

FIG. 6A is a diagram schematically showing examples of time variations in cerebral blood flow rate. As shown in FIG. 6A, the target part 10t of the user 10 is irradiated with light from the light source 20, and light returning from the target part 10t is detected. In this case, the surface-reflected component I1 is much larger than the internally-scattered component I2. However, the aforementioned shutter adjustment makes it possible to extract only the internally-scattered component I2. The graph shown in FIG. 6A shows changes in concentration of oxyhemoglobin (HbO₂) and deoxyhemoglobin (Hb) in cerebral blood with passage of time. In this example, the internally-scattered component I2 is acquired through the use of two wavelengths of light. The concentration shown in FIG. 6A represents an amount of change with reference to a normal amount. This amount of change is calculated by the signal processing circuit 70 on the basis of the signal strength of light. A change is observed in cerebral blood flow rate according to a state of brain activity such as a normal state, a concentrated state, or a relaxed state. There are variations in brain activity or variations in absorption coefficient or scattering coefficient from place to place in the target part 10t. For this reason, measurements may be performed at the same location in the target part 10t of the user 10. In a case where a change in brain activity with passage of time is observed, a state of the subject can be estimated from a temporal relative change in cerebral blood flow even when an absolute amount of cerebral blood flow is unknown.

It is not always necessary to use two wavelengths of light to detect a change in brain activity. In a case where one wavelength of light is used, it is possible to use light of any wavelength falling within a range of, for example, 810 nm to 880 nm. Light in this wavelength range is easily absorbed by HbO₂. In terms of a change in blood flow rate in the case of a change in state of brain activity, HbO₂ is often greater than Hb. Therefore, it is possible to read a trend of change in brain activity simply by making measurements with light in a wavelength range in which the optical absorptance of HbO₂ is high.

FIG. 6B is a diagram schematically showing an example of a case where measurements are simultaneously performed at a plurality of places within the target part 10t of the user 10. In this example, two-dimensional irradiation or two-dimensional imaging makes it possible to acquire a two-dimensional distribution of cerebral blood flow rate. In this case, the irradiation pattern of the light source 20 may for example be an uniform distribution of uniform intensity, a dot-shaped distribution, or a doughnut-shaped distribution. Irradiation with a uniform distribution of uniform intensity can make it unnecessary or easy to effect irradiation positioning on the target part 10t. Further, irradiation with a uniform distribution allows light to fall on the target part 10t of the user 10 from a wide range. This makes it possible to intensify a signal that is detected by the photodetector 30. Furthermore, this makes it possible to make measurements at any location within the irradiated region. A dot-shaped distribution or a doughnut-shaped distribution makes it possible to reduce the influence of the surface-reflected component I1 simply by removing the target part 10t from the irradiated region.

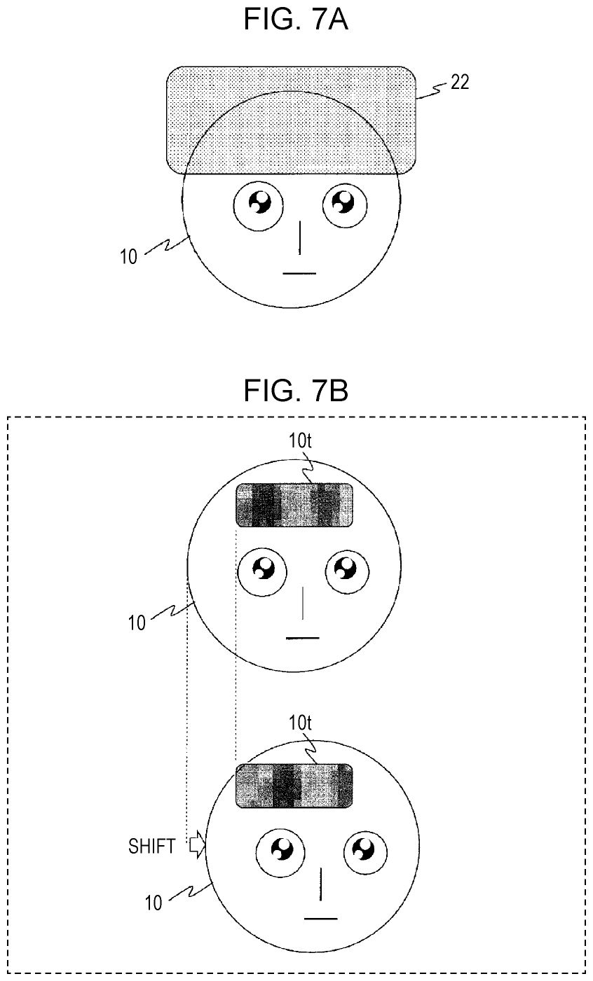

FIG. 7A is a diagram schematically showing an example of a region 22 that is irradiated with light. In noncontact cerebral blood flow measurement, the amount of light detected is attenuated in inverse proportion to the square of the distance from a device to a target part. Accordingly, a signal of each pixel that is detected by the photodetector 30 may be intensified by integrating signals of a plurality of nearby pixels. This makes it possible to reduce an integrated pulse number while maintaining the SN ratio. This brings about improvement in frame rate.

FIG. 7B is a diagram schematically showing a change in signal in the case of a lateral shift of the target part 10t of the user 10. As mentioned above, a change in brain activity can be read by detecting a difference between cerebral blood flow rate at the time of a change in state of brain activity from a normal state and cerebral blood flow rate in a normal state. In a case where a photodetector 30 including a plurality of photoelectric conversion elements arrayed two-dimensionally is used, a two-dimensional brain activity distribution can be acquired as shown in an upper portion of FIG. 7B. In this case, an active site of brain activity can be detected from a relative intensity distribution within the two-dimensional distribution even without acquiring a signal in a normal state in advance. In the present embodiment, in which measurements are performed in a noncontact manner, there may be a change in position of the target part 10t during measurement as shown in a lower portion of FIG. 7B. This may occur, for example, in a case where the user 10 has moved in order to breath. In general, a two-dimensional distribution of cerebral blood flow rate does not abruptly change within a very short time. For this reason, the displacement of the target part 10t can be corrected, for example, by pattern matching between frames of two-dimensional distribution detected. Alternatively, in the case of periodic movement such as breathing, only a frequency component of the movement may be extracted for correction or elimination. The target part 10t does not need to be a single region but may be a plurality of regions. The plurality of regions may include, for example, one right region and one left region or may be distributed in a 2×6 matrix of dots.

5. Example of Signal Processing that Reduces Influence of Motion of User During Measurement

The following describes a control method for detecting the internally-scattered component I2 with high accuracy even in a case where the user 10 has moved back and forth during measurement. In the following description, a forward or backward motion of the user 10 is expressed as “body motion”.

In the example shown in FIG. 1A, a body motion of the user 10 during measurement may effect a change in the distance from the optical measuring device 100 to the user 10. Performing the aforementioned process with disregard to this change in distance may make it impossible to correctly detect the internally-scattered component I2.

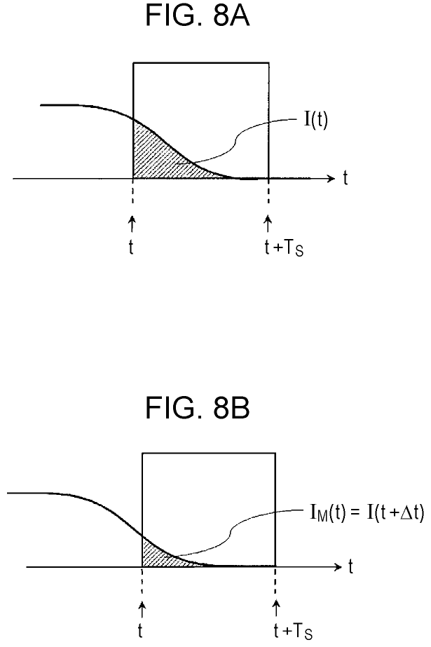

FIG. 8A is a diagram schematically showing an example of a rear-end component of a reflected light pulse that is detected in a case where the target part 10t of the user 10 is at a predetermined distance from the device. FIG. 8B is a diagram schematically showing an example of a rear-end component of a reflected light pulse that is detected in a case where the target part 10t of the user 10 has moved nearer to the device during measurement. The rectangles in FIGS. 8A and 8B each represent an exposure period of a particular time length Tₛ. In this example, the origin of the time axis represents the time point at which the emission of each light pulse is started, and an exposure continues from a time t to a time t+Tₛ. Assume that I(t) is the integrated amount of light of a reflected light pulse that is detected in the absence of a body motion of the user 10. Assume that the I_{M}(t) is the integrated amount of light of a reflected light pulse that is detected in the presence of a body motion of the user 10. The integrated amount of light refers to the amount of light of a reflected light pulse having arrived during a period from the time t to the time t+Tₛ. That is, the integrated amount of time is equivalent to the total value of detection signals during an exposure period of the time length Tₛ. Further, the integrated amount of light can also be calculated by integrating the intensity of a reflected light pulse from the time t to the time t+Tₛ. The shaded areas shown in FIGS. 8A and 8B are each equivalent to the integrated amount of light.

As shown in FIGS. 8A and 8B, a shortening of the distance to the target part 10t causes the integrated amount of light I_{M}(t) to become smaller than the integrated amount of light I(t). This is attributed to the arrival of a reflected light pulse at the photodetector 30 at an earlier timing. When a body motion causes a reflected light pulse to arrive at an earlier timing by Δt, the relationship I_{M}(t)=I(t+Δt) holds. When the distance to the target part 10t becomes shorter, Δt>0, and when the distance to the target part 10t becomes longer, Δt<0. In a case where the distance to the target part 10t becomes longer, the integrated amount of light I_{M}(t) becomes larger than the integrated amount of light I(t). A fluctuation in integrated amount of light due to a body motion of the user 10 may cause a decrease in accuracy of measurement of the internally-scattered component I2.

The optical measuring device 100 according to the present embodiment measures the internally-scattered component I2 by combining, for one reflected light pulse, two signals acquired in two different exposure periods. More specifically, the control circuit 60 executes the following operations.

(1) The control circuit 60 causes the light source 20 to emit a first light pulse and a second light pulse at different timings.
(2) The control circuit 60 causes the photodetector 30 to output a first signal representing an amount of light of a component of a first reflected light pulse attributed to the first light pulse, the component having arrived during an exposure period of a particular time length from a first time point at which a first period of time has elapsed since the start of a fall time of the first reflected light pulse.
(3) The control circuit 60 causes the photodetector 30 to output a second signal representing an amount of light of a component of a second reflected light pulse attributed to the second light pulse, the component having arrived during an exposure period of a particular time length from a second time point at which a second period of time has elapsed since the start of a fall time of the second reflected light pulse.

The signal processing circuit 70 generates cerebral blood flow information on the user 10 by executing a computation involving the use of the first signal and the second signal.

Such an operation makes it possible to more accurately detect the internally-scattered component I2 even in a case where the user 10 has moved back and forth.

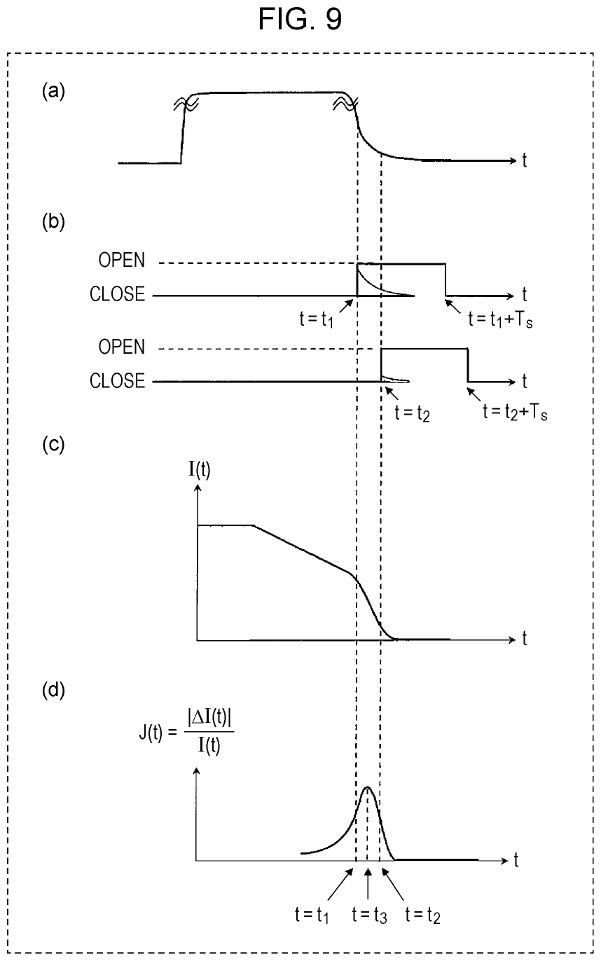

FIG. 9 is a diagram explaining the principle of measurement by an exemplary embodiment.

A portion (a) of FIG. 9 schematically shows an example of a time response waveform of an optical signal of a reflected light pulse that arrives at the photodetector 30. A portion (b) of FIG. 9 schematically shows a situation where a portion of a rear-end component of the reflected light pulse is detected in two different exposure periods. In the example shown in the portion (b) of FIG. 9, the integrated amount of light I(t₁) of the reflected light pulse from t=t₁ to t=t₁+Tₛ, and the integrated amount of light I(t₂) of the reflected light pulse from t=t₂ to t=t₂+Tₛ. The relationship between t₁ and t₂ will be described later. A portion (c) of FIG. 9 schematically shows time dependency of the integrated amount of light I(t) of the reflected light pulse from the time t to the time t+Tₛ. A portion (d) of FIG. 9 schematically shows time dependency of a function J(t) obtained by dividing, by the integrated amount of light I(t), the absolute value of the amount of change ΔI(t) in the integrated amount of light I(t) due to a body motion of the user 10. ΔI(t)=μm(t)−I(t)|. J(t) is expressed by Formula (1) as follows:

$$J ( t ) = \frac{\vert Δ I ( t ) \vert}{I ( t )} = \frac{\vert {I}_{M} ( t ) - I ( t ) \vert}{I ( t )} \qquad ( 1 )$$

J(t) reaches its maximum at t=t₃. t₁ is a time point that precedes t₃, and t₂ is a time point that follows t₃. Since the speed of a body motion of the user 10 is much slower than the speed of light, Δt in the integrated amount of light I_{M}(t)=I(t+Δt) is sufficiently small. Accordingly, the integrated amount of light I_{M}(t)=I(t+Δt) is approximated to Formula (2) as follows:

$${I}_{M} ( t ) = I ( t + Δ t ) \approx I ( t ) + \frac{dI ( t )}{dt} Δ t \qquad ( 2 )$$

Substituting Formula (2) in Formula (1) causes J(t) to be approximated to Formula (3) as follows:

$$J ( t ) \approx \frac{1}{I ( t )} \vert \frac{dI ( t )}{dt} \vert \vert Δ t \vert \qquad ( 3 )$$

The time dependency of J(t) shown in the portion (d) of FIG. 9 is obtained from Formula (3). In the following discussion, it is not necessary to find a specific value of Δt.

In the example shown in the portion (d) of FIG. 9, t₁ and t₂ may be set to satisfy J(t₁)=J(t₂). J(t₁)=J(t₂) is expressed by Formula (4) as follows:

$$\vert \frac{{I}_{M} ( {t}_{1} )}{I ( {t}_{1} )} - 1 \vert = \vert \frac{{I}_{M} ( {t}_{2} )}{I ( {t}_{2} )} - 1 \vert \qquad ( 4 )$$

Transforming I_{M}(t₁)/I(t₁)=I_{M}(t₂)/I(t₂) obtained from Formula (4) gives Formula (5) as follows:

$$R = \frac{I ( {t}_{2} )}{I ( {t}_{1} )} = \frac{{I}_{M} ( {t}_{2} )}{{I}_{M} ( {t}_{1} )} \qquad ( 5 )$$

In Formula (5), the ratio between I(t₁) and I(t₂) in the absence of a body motion of the user 10 and the ratio between I_{M}(t₁) and I_{M}(t₂) in the presence of a body motion of the user 10 are the same. Accordingly, setting t₁ and t₂ to satisfy J(t₁)=J(t₂) shows that the ratio R between the integrated amount of light I(t₁) and the integrated amount of light I(t₂) is constant regardless of a body motion of the user 10. In other words, when the internal state of the target part 10t of the user 10 is constant, the ratio R in a case where the distance to the target part 10t of the user 10 is a first distance is equal to the ratio R in a case where the distance to the target part 10t of the user 10 is a second distance that is different from the first distance. In this way, the ratio R is noted affected by a body motion of the user 10.

The ratio R calculated by division is not an absolute value but a relative value. Accordingly, time dependency of the ratio R obtained by frame-by-frame measurement is effective, for example, in examining the extent of a change in cerebral blood flow in the head of the user 10 over time since the start of the measurement. The time dependency of the ratio R may be normalized to R/Ro by the ratio R at the start of the measurement. Finding a specific value of cerebral blood flow at the start of the measurement makes it possible to find specific time dependency of cerebral blood flow by multiplying R/Ro by the value.

Even in a case where J(t₁)≠J(t₂), it is expected that the ratio R is not greatly affected by a body motion of the user 10, provided the difference between J(t₁) and J(t₂) is modest. If a value obtained by dividing the amount of change ΔR in the ratio R due to a body motion of the user 10 by the ratio R falls, for example, within a range of |ΔR|/R<0.2, the ratio R may be said to be substantially equal regardless of the body motion of the user 10. Furthermore, even in a case where J(t₁)≠J(t₂), it is possible, for example, to assign weights and/or add correction values to I(t₁) and I(t₂), respectively, in the ratio (R) so that J(t) becomes equal.

The following describes how the optical measuring device 100 according to the present embodiment operates in acquiring cerebral blood flow information on the user 10 on the basis of the foregoing principle.

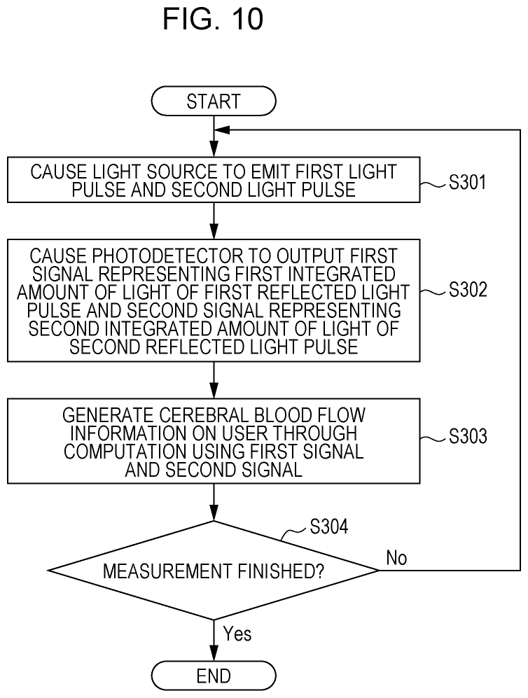

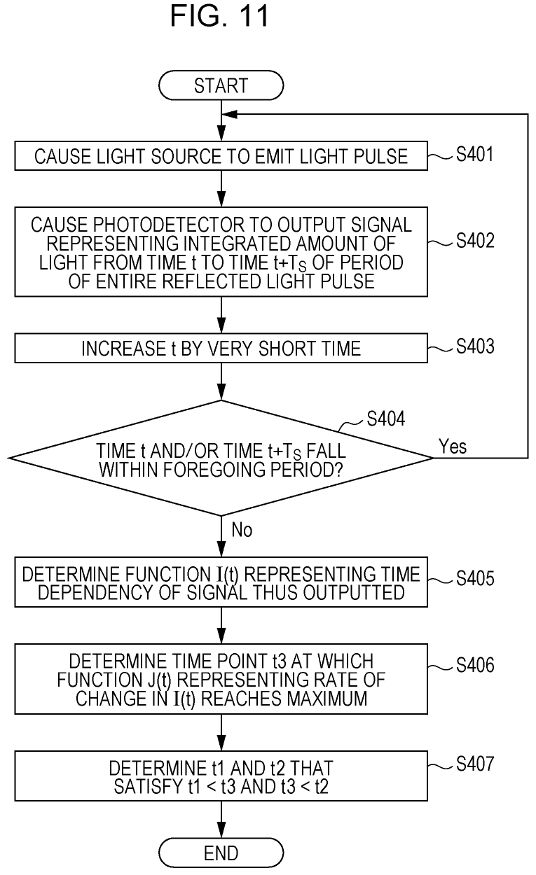

FIG. 10 is a flow chart showing an example of operation of the optical measuring device 100 in an exemplary embodiment. The distance between the target part 10t of the user 10 and the photodetector 30 is a predetermined distance. This distance falls within a range of distances supposed in advance to be an appropriate range of distances between the target part 10t of the user 10 and the photodetector 30 during the use of the optical measuring device 100. In the following description, a first time point at which a first period of time has elapsed since the start of a fall time of a reflected light pulse is equivalent to t₁ shown in FIG. 9, and a second time point at which a second period of time that is longer the first period of time has elapsed since the start the fall time of the reflected light pulse is equivalent to t₂ shown in FIG. 9.

In step S301, the control circuit 60 causes the light source 20 to emit a first light pulse and a second light pulse at different timings. Due to the first light pulse and the second light pulse, a first reflected light pulse and a second reflected light pulse each return from the head of the user 10 to the photodetector 30.

In step S302, the control circuit 60 causes the photodetector 30 to detect a first integrated amount of light from t=t₁ to t=t₁+Tₛ in a fall time of the first reflected light pulse and output a first signal representing the first integrated amount of light and causes the photodetector 30 to detect a second integrated amount of light from t=t₂ to t=t₂+Tₛ in a fall time of the second reflected light pulse and output a second signal representing the second integrated amount of light.

The fall time of the first reflected pulse is herein sometimes referred to as “first fall time”, and the fall time of the second reflected pulse is herein sometimes referred to as “second fall time”. Further, a portion from t=t₁ to t=t₁+Tₛ in the fall time of the first reflected light pulse is sometimes referred to as “first portion”, and a portion from t=t₂ to t=t₂+Tₛ in the fall time of the second reflected light pulse is sometimes referred to as “second portion”. Further, an exposure period from t=t₁ to t=t₁+Tₛ of the first reflected pulse is sometimes referred to as “first period having a first time length”, and an exposure period from t=t₂ to t=t₂+Tₛ of the second reflected pulse is sometimes referred to as “second period having a second time length”. A time interval from a start of the first fall time to the first time point is different from a time interval from a start of the second fall time to the second time point. Although, in the example shown in FIG. 9, the first time length and the second time length are identical to each other, they may be different from each other to the extent that the foregoing principle is valid.

The control circuit 60 may cause the photodetector 30 to output the first signal and the second signal after having caused the photodetector 30 to detect the first integrated amount of light and the second integrated amount of light. Alternatively, the control circuit 60 may cause the photodetector 30 to output the first signal after having caused the photodetector 30 to detect the first integrated amount of light and then cause the photodetector 30 to output the second signal after having causes the photodetector 30 to detect the second integrated amount of light. Alternatively, the control circuit 60 may cause the photodetector 30 to output the second signal after having caused the photodetector 30 to detect the second integrated amount of light and then cause the photodetector 30 to output the first signal after having causes the photodetector 30 to detect the first integrated amount of light.

In detection of a reflected light pulse, as shown in the portion (b) of FIG. 9, the exposure period from t=t₁ to t=t₁+Tₛ and the exposure period from t=t₂ to t=t₂+Tₛ generally overlap each other. In a period of overlap between the two exposure periods, signal charge corresponding to the first integrated amount of light of one reflected light pulse and signal charge corresponding to the second integrated amount of light of the reflected light pulse cannot be accumulated in different signal accumulators in the same pixel. For this reason, the first integrated amount of light and the second integrated amount of light are detected not from one reflected light pulse but from the first reflected light pulse and the second reflected light pulse, respectively, in the same pixel.

In step S303, the signal processing circuit 70 generates cerebral blood flow information on the user 10 by executing a computation involving the use of the first signal and the second signal.

In step S304, the signal processing circuit 70 determines whether the measurement has finished. In a case where the signal processing circuit 70 determines that the measurement has not finished, the control circuit 60 and the signal processing circuit 70 repeat steps S301 to S303 until the signal processing circuit 70 determines that the measurement has finished. The determination as to whether the measurement has finished may be made, for example, on the basis of whether there has been a stop order from the user 10. Alternatively, the determination as to whether the measurement has finished may be made on the basis of whether a predetermined period of time has elapsed since the start of the measurement or whether a predetermined amount of data has been accumulated since the start of the measurement.

This makes it possible to generate, on the basis of a fluctuation in the first signal and a fluctuation in the second signal, information indicating a fluctuation in cerebral blood flow on the user 10. As shown in Formula (5), even when the distance between the target part 10t of the user 10 and the photodetector 30 changes due to a body motion of the user 10, a computed value obtained by dividing the value of either one of the first and second signals by the value of the other one of the first and second signals is substantially equal to a computed value obtained in a case where the distance is a predetermined signal. From this computed value, the cerebral blood flow information on the user 10 is generated.

The control circuit 60 may repeatedly execute steps S301 and S302. In this case, a plurality of first reflected light pulses attributed to a plurality of first light pulses emitted from the light source 20 return from the head of the user 10 to the photodetector 30. Similarly, a plurality of second reflected light pulses attributed to a plurality of second light pulses emitted from the light source 20 return from the head of the user 10 to the photodetector 30. The first signal represents the total of first integrated amounts of light from t=t₁ to t=t₁+Tₛ in fall times of the plurality of first reflected light pulses. Similarly, the second signal represents the total of second integrated amounts of light from t=t₂ to t=t₂+Tₛ in fall times of the plurality of second reflected light pulses.

In repeating steps S301 and S302, the control circuit 60 may cause the light source 20 to alternately emit the first light pulse and the second light pulse more than once. Alternatively, the control circuit 60 may cause the light source 20 to emit the second light pulse more than once after having caused the light source 20 to emit the first light pulse more than once or cause the light source 20 to emit the first light pulse more than once after having caused the light source 20 to emit the second light pulse more than once. The number of times the first light pulse is emitted and the number of times the second light pulse is emitted may be the same as or different from each other. In a case where the numbers of times are different from each other, the difference in number of times may be corrected in the computation in step S303 involving the use of the first signal and the second signal.

t₁ and t₂ are adjusted by a calibration operation prior to the start of a measurement. The following describes how the optical measuring device 100 according to the present embodiment operates before the start of a measurement.

FIG. 11 is a flow chart showing an example of operation of the optical measuring device 100 before the start of a measurement. Prior to execution of this operation, the distance between the target part 10t of the user 10 and the photodetector 30 is measured, for example, by the method shown in FIG. 4. Assume that this distance is an appropriate distance between the target part 10t of the user 10 and the photodetector 30 during the use of the optical measuring device 100. In the following description, a time point at which a third period of time has elapsed since the start point of a fall time of a reflected light pulse is equivalent to t₃ shown in FIG. 9.

In step S401, the control circuit 60 causes the light source 20 to emit a light pulse. Due to the light pulse, a reflected light pulse returns from the head of the user 10 to the photodetector 30.

In step S402, the control circuit 60 causes the photodetector 30 to output a signal representing an integrated amount of light I(t) from a time t to a time t+Tₛ in a period from the start of an increase in intensity of the reflected light pulse to the end of a decrease in intensity of the reflected light pulse. The period from the start of the increase in intensity of the reflected light pulse to the end of the decrease is a period from the start to the end of incidence of the whole reflected light pulse on the photodetector 30.

In step S403, the control circuit 60 increases t by a very short time δt (>0). The very short time δt ranges, for example, from several tens of picoseconds to several tens of nanoseconds. It should be noted that the very short time δt has no relationship with Δt in the integrated amount of light I_{M}(t)=I(t+Δt).

In step S404, the control circuit 60 determines whether the time t and/or the time t+Tₛ fall(s) within the period from the start of the increase in intensity of the reflected light pulse to the end of the decrease. It should be noted that prior to the start of the operation shown in FIG. 11, the time t+Tₛ may be set to coincide with a time point at which the increase in intensity of the reflected light pulse starts. Meanwhile, the computation of Formula (3) involves the use of time dependency of the integrated amount of light I(t) near the rear end of the reflected light pulse. Accordingly, prior to the start of the operation shown in FIG. 11, the time t+Tₛ may be set to coincide with the start point of the fall time of the reflected light pulse. Instead of the time t+Tₛ, the time t may be set to coincide with the start point of the fall time of the reflected light pulse. If Yes to the determination in step S404, the control circuit 60 returns to step S401. If No to the determination in step S404, the control circuit 60 proceeds to step S405.

By repeating steps S401 to S403, the control circuit 60 continuously executes the operation shown in steps S401 and S402 while shifting the time t, which is a starting point of an exposure period from the time t to the time t+Tₛ, by the very short time δt. The control circuit 60 may be said to execute the following operation. That is, the control circuit 60 causes the light source 20 to emit a plurality of light pulses and causes the photodetector 30 to detect a plurality of reflected light pulses attributed to the plurality of light pulses while shifting, by the very short time, the time lag between the start of a decrease in intensity of each of the plurality of reflected light pulses to the start of detection. The time length of an exposure time from the time t to the time t+Tₛ in the repetition of steps S401 to S403 is herein sometimes referred to as “third time length”. The third time length, which is identical to the first time length and the second time length, may be different from the first time length and the second time length to the extent that the foregoing principle is valid.

In step S405, the control circuit 60 acquires the integrated amount of light I(t) of the reflected light pulses from time dependency of signals acquired by repeating steps S401 to S403. This time dependency is obtained by discrete sampling of the very short time δt.

In step S406, the signal processing circuit 70 calculates a third period of time at which J(t) in Formula (3) reaches its maximum. In the computation of Formula (3), dl(t)/dt is approximated to dl(t)/dt≈[I(t+δt)−I(t)]/δt, for example, by subtraction. This causes Formula (3) to be approximated to Formula (6) as follows:

$$J ( t ) \approx \frac{\vert I ( t + δ t ) - I ( t ) \vert}{I ( t )} \frac{\vert Δ t \vert}{δ t} \qquad ( 6 )$$

In Formula (6), it is not necessary to find a specific value of |Δt|/δt. The signal processing circuit 70 calculates, from the starting point of a fall time of each light pulse during repetition of steps S401 to S403, a third period of time at which |I(t+δt)−I(t)|/I(t) obtained by dividing, by I(t), the absolute value |I(t+t)−I(t)| of the amount of change for the very short time δt in I(t), which represents time dependency on the time at which the signal originates, reaches its maximum. The time point at which the third period of time has elapsed since the start point of the first fall time of the first reflected pulse is herein sometimes referred to as “third time point”, and the time point at which the third period of time has elapsed since the start point of the second fall time of the second reflected pulse is herein sometimes referred to as “fourth time point”. It should be noted that |Δt|/δt may be omitted from Formula (6) so that J(t)=|I(t+δt)−I(t)|/I(t).

In step S407, the control circuit 60 sets the first period of time to be shorter than the third period of time and sets the second period of time longer than the third period of time. That is, t₁<t₃, and t₂>t₃. t₁<t₃ can be construed as meaning that the first time point precedes the third time point. t₂>t₃ can be construed as meaning that the second time point follows the fourth time point. When J(t) takes on the same value at t=t¹, which is a time point at which the first period of time has elapsed since the start of the fall time of the light pulse, and t=t₂, which is a time point at which the second period of time has elapsed, J(t₁)=J(t₂) is satisfied. On this occasion, the ratio R in Formula (5) makes it possible to minimize a decrease in measurement accuracy caused by a body motion of the user 10.

The optical measuring device 100 according to the present embodiment is supposed to be used in a state where the distance between the target part 10t of the user 10 and the photodetector 30 falls within a predetermined range of distances. For example, in a case where there is a chair or a seat in front of the photodetector 30 and the distance between the photodetector 30 and the chair or the seat is fixed, the distance between the target part 10t of the user 10 and the photodetector 30 is considered to be almost the same every time, provided the user 10 sits on the chair or the seat in the same position every time. Accordingly, once t₁ and t₂ are set by steps S401 to S407 shown in FIG. 11 before the start of a measurement, steps S301 to S303 shown in FIG. 10 can be executed using the same t₁ and t₂ from the second and subsequent times.

Further, in a case where the distance varies from measurement to measurement, t₁ and t₂ may be periodically reset by steps S401 to S407 shown in FIG. 11.

Alternatively, in a case where a separately-provided distance sensor has sensed a great fluctuation in the distance between the target part 10t of the user 10 and the photodetector 30, t₁ and t₂ may be reset by steps S401 to S407 shown in FIG. 11.

The following describes a modification of the optical measuring device 100 according to the present embodiment. In the aforementioned example, the first integrated amount of light and the second integrated amount of light are detected from the first reflected light pulse and the second light reflected pulse, respectively. In the following modification, the first integrated amount of light and the second integrated amount of light are detected from one reflected light pulse. The photodetector 30 here is an image sensor including a plurality of pixels. Each pixel outputs a signal representing the amount of light of at least a portion of a reflected light pulse returning from the head of the user 10.

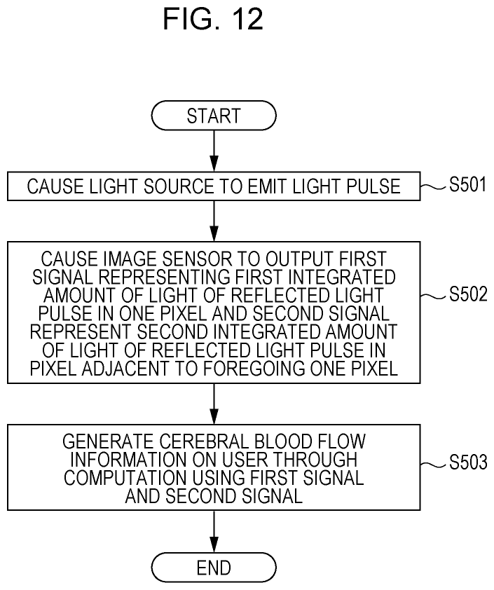

FIG. 12 is a flow chart showing a first modification of operation of the optical measuring device 100. The control circuit 60 may execute steps S501 to S503 shown in FIG. 12 instead of executing steps S301 to S303 shown in FIG. 10.

In step S501, the control circuit 60 causes the light source 20 to emit a light pulse. Due to the light pulse, a reflected light pulse returns from the heat of the user 10 to the image sensor.

In step S502, the control circuit 60 causes the image sensor to detect a first integrated amount of light from t=t₁ to t=t₁+Tₛ in a fall time of a reflected light pulse in one pixel of the plurality of pixels and causes the image sensor to output a first signal representing the first integrated amount of light. Furthermore, the control circuit 60 causes the image sensor to detect a second integrated amount of light from t=t₂ to t=t₂+Tₛ in the fall time of the reflected light pulse in a pixel adjacent to the one pixel of the plurality of pixels and causes the image sensor to output a second signal representing the second integrated amount of light. The waveforms of reflected light pulses that fall on two adjacent pixels of the plurality of pixels are considered to be almost the same. This makes it possible to obtain the first integrated amount of light and the second integrated amount of light from the same one reflected light pulse. The control circuit 60 causes the image sensor to output the first signal and the second signal after having caused the image sensor to detect the first integrated amount of light and the second integrated amount of light. A portion from t=t₁ to t=t₁+Tₛ in the fall time of the reflected light pulse is herein sometimes referred to as “first portion”, and a portion from t=t₂ to t=t₂+Tₛ in the fall time of the reflected light pulse is herein sometimes referred to as “second portion”. Further, an exposure period from t=t₁ to t=t₁+Tₛ of the reflected pulse is sometimes referred to as “first period having a first time length”, and an exposure period from t=t₂ to t=t₂+Tₛ of the reflected pulse is sometimes referred to as “second period having a second time length”. Although the first time length and the second time length are identical to each other, they may be different from each other to the extent that the foregoing principle is valid.

The control circuit 60 may repeatedly execute steps S501 and S502. In this case, a plurality of reflected light pulses attributed to a plurality of light pulses emitted from the light source 20 return from the head of the user 10 to the photodetector 30. The first signal represents the total of first integrated amounts of light from t=t₁ to t=t₁+Tₛ in fall times of the plurality of reflected light pulses in one pixel of the plurality of pixels. Similarly, the second signal represents the total of second integrated amounts of light from t=t₂ to t=t₂+Tₛ in fall times of the plurality of reflected light pulses in a pixel adjacent to the one pixel of the plurality of pixels.

Step S503 is identical in operation to step S303.

In the aforementioned example, the exposure period from t=t₁ to t=t₁+Tₛ and the exposure period from t=t₂ to t=t₂+Tₛ overlap each other. For this reason, the first integrated amount of light and the second integrated amount of light cannot be detected from one reflected light pulse in the same pixel. Meanwhile, when the exposure period from t=t₁ to t=t₁+Tₛ and the exposure period from t=t₂ to t=t₂+Tₛ do not overlap each other, the first integrated amount of light and the second integrated amount of light can be detected from one reflected light pulse in the same pixel.

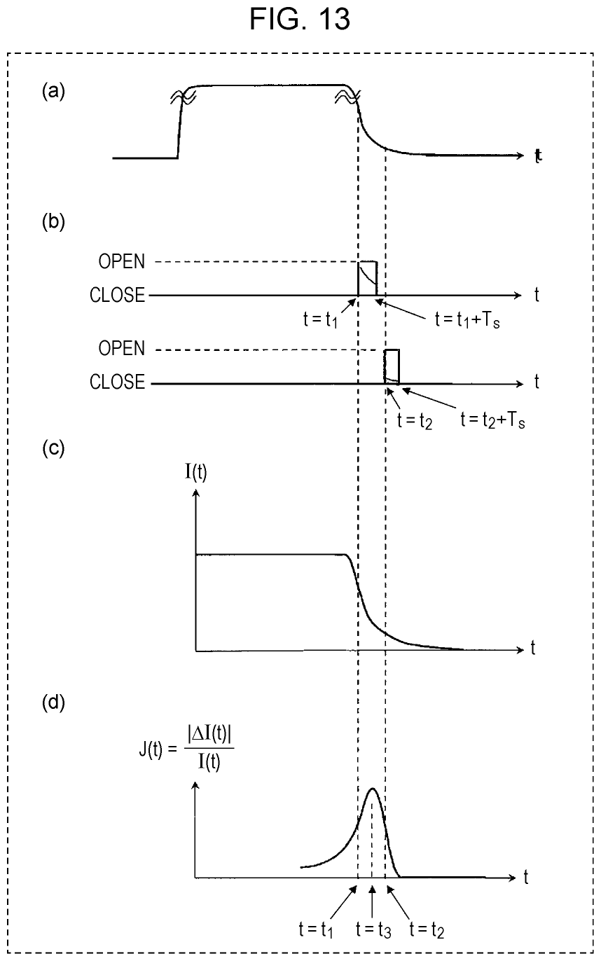

FIG. 13 is another diagram explaining the principle of measurement.

A portion (a) of FIG. 13 schematically shows an example of a time response waveform of an optical signal of a reflected light pulse that arrives at the photodetector 30. A portion (b) of FIG. 13 schematically shows a situation where a portion of a rear-end component of the reflected light pulse is detected in two different exposure periods. A portion (c) of FIG. 13 schematically shows time dependency of the integrated amount of light I(t) of the reflected light pulse from the time t to the time t+Tₛ. A portion (d) of FIG. 13 schematically shows time dependency of a function J(t).

As shown in the portion (b) of FIG. 13, the exposure period from t=t₁ to t=t₁+Tₛ and the exposure period from t=t₂ to t=t₂+Tₛ do not overlap each other. In this case, the time dependency of the integrated amount of light I(t) of the reflected light pulse shown in the portion (c) of FIG. 13 is different from the time dependency of the integrated amount of light I(t) of the reflected light pulse shown in the portion (c) of FIG. 9. However, the function J(t) shown in the portion (d) of FIG. 13 is similar to the function J(t) shown in the portion (d) of FIG. 9. Accordingly, according to the aforementioned principle, setting t₁ and t₂ to satisfy J(t₁)=J(t₂) causes the ratio R between the integrated amount of light I(t₁) and the integrated amount of light I(t₂) to be constant regardless of a body motion of the user 10.

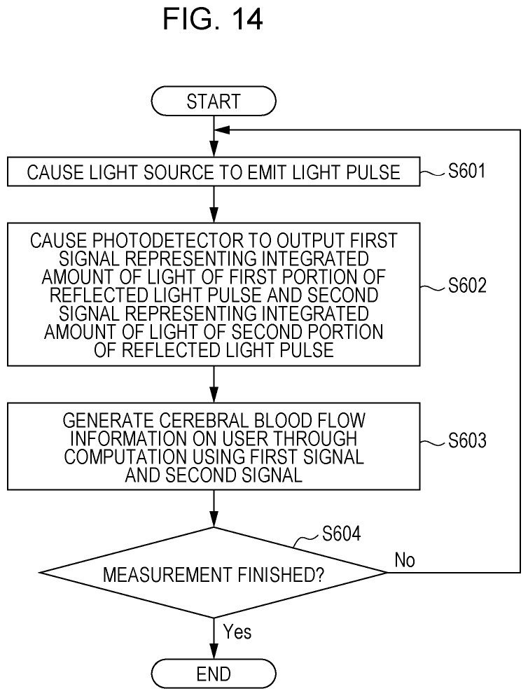

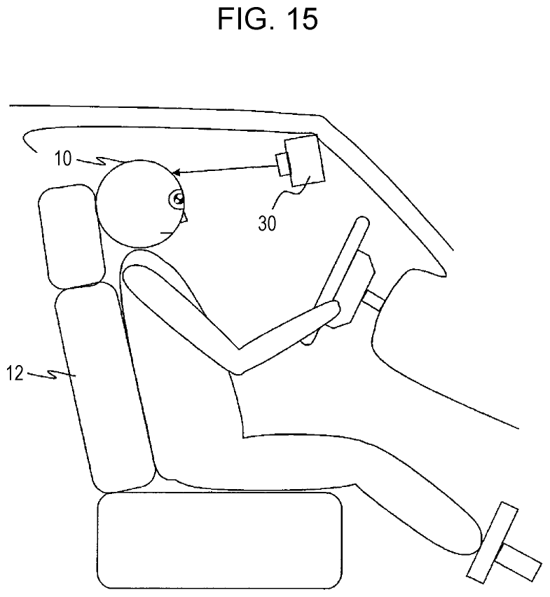

FIG. 14 is a flow chart showing a second modification of operation of the optical measuring device 100. The control circuit 60 may execute steps S601 to S604 shown in FIG. 14 instead of executing steps S301 to S304 shown in FIG. 10. Contents which overlap those of the flow chart shown in FIG. 10 are omitted.

In step S601, the control circuit 60 causes the light source 20 to emit a light pulse. Due to the light pulse, a reflected light pulse returns from the heat of the user 10 to the photodetector 30.

In step S602, the control circuit 60 causes the photodetector 30 to detect a first integrated amount of light from t=t₁ to t=t₁+Tₛ in a fall time of the reflected light pulse and output a first signal representing the first integrated amount of light and causes the photodetector 30 to detect a second integrated amount of light from t=t₂ to t=t₂+Tₛ in the fall time of the reflected light pulse and output a second signal representing the second integrated amount of light.

Step S603 is identical in operation to step S303.

In step S604, the signal processing circuit 70 determines whether the measurement has finished. In a case where the signal processing circuit 70 determines that the measurement has not finished, the control circuit 60 and the signal processing circuit 70 repeat steps S601 to S603 until the signal processing circuit 70 determines that the measurement has finished.

The following describes an example of application of the optical measuring device 100 according to the present embodiment.

FIG. 15 is a diagram schematically showing an example of acquisition of cerebral blood flow information on a user 10 sitting on a seat 12 in an automobile. In the example shown in FIG. 15, the photodetector 30 of the optical measuring device 100 measures the cerebral blood flow information on the user 10 during driving of the automobile. Measuring the cerebral blood flow information makes it possible to check whether the user 10 is in a careless state or whether the user 10 may have an accident. During the measurement of the cerebral blood flow information, a body motion of the user 10 occurs due to a vibration of the car body, so that the distance between the target part 10t of the user 10 and the photodetector 30 may change. Even in this case, because of the ratio R in Formula (5), the optical measuring device 100 according to the present embodiment prevents the cerebral blood flow information on the user 10 from being greatly affected by the body motion of the user 10. Accordingly, applying the optical measuring device 100 according to the present embodiment to monitoring for automatic driving and/or drive assist makes it possible to measure the cerebral blood flow information on the user 10 with high accuracy.

The present disclosure also encompasses a method and program for an operation that the control circuit 60 and the signal processing circuit 70 execute.

The embodiment described above has illustrated a case where the target of measurement by the optical measuring device 100 is cerebral blood flow information on a human body. However, the target of measurement by the optical measuring device 100 is not limited to cerebral blood flow information, and the optical measuring device 100 can also be applied to measurement of blood flow information on a comparatively deep part other than the brain. Further, the optical measuring device 100 can also be applied to an object whose internal state fluctuates over time.

An optical measuring device according to the present embodiment can be utilized, for example, for diagnosing a mental state such as a degree of concentration during work when a particular user performs particular work in a particular place. Further, an optical measuring device according to the present embodiment can be applied, for example, to periodic diagnosis of mental illness in a hospital, diagnosis of a metal state in a brain training gym, detection of a degree of concentration or a degree of difficulty of a task during desk work, or prediction of an error or detection of a careless state during the work of operating an apparatus.

What is claimed is:

1. An optical measuring device comprising:
a light source that emits light pulses with which a target of measurement is irradiated;
a photodetector that detects at least a portion of each of reflected light pulses returning from the target of measurement;
a control circuit that controls the light source and the photodetector; and
a signal processing circuit that processes a signal outputted from the photodetector, wherein
the light pulses include a first light pulse and a second light pulse,
the reflected light pulses include a first reflected light pulse attributed to the first light pulse and a second reflected light pulse attributed to the second light pulse,
the control circuit causes the light source to emit the first light pulse and the second light pulse at different timings, respectively,
the control circuit causes the photodetector to detect a first portion of the first reflected light pulse in a first period having a first time length and output a first signal representing an amount of light of the first portion, the first period starting from a first time point during a first fall time that is a period from a start to an end of a decrease in intensity of the first reflected light pulse,
the control circuit causes the photodetector to detect a second portion of the second reflected light pulse in a second period having a second time length and output a second signal representing an amount of light of the second portion, the second period starting from a second time point during a second fall time that is a period from a start to an end of a decrease in intensity of the second reflected light pulse,
a time interval from a start of the first fall time to the first time point is different from a time interval from a start of the second fall time to the second time point,
the control circuit executes first control more than once, the first control including causing the light source to emit the first light pulse, causing the photodetector to detect the first reflected light pulse, and causing the photodetector to output the first signal,
the control circuit executes second control more than once, the second control including causing the light source to emit the second light pulse, causing the photodetector to detect the second reflected light pulse, and causing the photodetector to output the second signal, and
the signal processing circuit generates, based on a fluctuation in the first signal and a fluctuation in the second signal, information indicating a fluctuation in internal state of the target of measurement.

2. The optical measuring device according to claim 1, wherein the first time length and the second time length are identical to each other.

3. The optical measuring device according to claim 2, wherein
the first time point precedes a third time point at which a value of J(t) represented by Formula (1) reaches maximum of the value in the first fall time,
the second time point follows a fourth time point at which the value of J(t) reaches maximum of the value in the second fall time,
$$J ( t ) = \frac{\vert I ( t + δ t ) - I ( t ) \vert}{I ( t )} \qquad ( 1 )$$
where t is a time to start to detect the first reflected light pulse or the second reflected light pulse, δt is a very short time, and I(t) is an amount obtained by integrating an amount of light of the first reflected light pulse detected in the first period or an amount obtained by integrating an amount of light of the second reflected light pulse detected in the second period.

4. The optical measuring device according to claim 1, wherein the signal processing circuit generates the information based on a fluctuation in ratio between the first signal and the second signal.

5. The optical measuring device according to claim 1, wherein when the internal state of the target of measurement is constant, a value of a ratio between the first signal and the second signal in a case where a distance between the target of measurement and the photodetector is a first distance is substantially equal to a value of the ratio in a case where the distance between the target of measurement and the photodetector is a second distance that is different from the first distance.

6. The optical measuring device according to claim 1, wherein
the target of measurement is a living organism, and
the information indicates a fluctuation in amount of blood flow of the target of measurement.

7. The optical measuring device according to claim 6, wherein the blood flow is cerebral blood flow of the living organism.

8. The optical measuring device according to claim 1, wherein
the control circuit causes the light source and the photodetector to execute a calibration operation of adjusting the first time point and the second time point,
in the calibration operation, the control circuit causes the light source to emit third light pulses and causes the photodetector to detect third reflected light pulses attributed to the third light pulses, the third reflected light pulses being detected with a very short time shift in time lag between a start of a decrease in intensity of each of the third reflected light pulse and a start of detection,
the third reflected light pulses are each detected in a period having a third time length, and
the first time length, the second time length, and the third time length are identical to one another.

9. An optical measuring device comprising:
a light source that emits a first light pulse with which a target of measurement is irradiated;
a photodetector that detects at least a portion of a first reflected light pulse returning from the target of measurement due to the first light pulse;
a control circuit that controls the light source and the photodetector; and
a signal processing circuit that processes a signal outputted from the photodetector, wherein
the control circuit causes the light source to emit the first light pulse,
the control circuit causes the photodetector to detect a first portion of the first reflected light pulse in a first period having a first time length and output a first signal representing an amount of light of the first portion, the first period starting from a first time point during a fall time that is a period from a start to an end of a decrease in intensity of the first reflected light pulse,
the control circuit causes the photodetector to detect a second portion of the first reflected light pulse in a second period having a second time length and output a second signal representing an amount of light of the second portion, the second period starting from a second time point during the fall time,
a time interval from a start of the fall time to the first time point is different from a time interval from the start of the fall time to the second time point,
the control circuit executes control more than once, the control including causing the light source to emit the first light pulse, causing the photodetector to detect the first reflected light pulse, and causing the photodetector to output the first signal and the second signal, and
the signal processing circuit generates, based on a fluctuation in the first signal and a fluctuation in the second signal, information indicating a fluctuation in internal state of the target of measurement.

10. The optical measuring device according to claim 9, wherein the first time length and the second time length are identical to each other.

11. The optical measuring device according to claim 10, wherein
the first time point precedes a third time point at which a value of J(t) represented by Formula (1) reaches maximum of the value in the fall time,
the second time point follows the third time point,
$$J ( t ) = \frac{\vert I ( t + δ t ) - I ( t ) \vert}{I ( t )} \qquad ( 1 )$$
where t is a time to start to detect the first reflected light pulse, of is a very short time, and I(t) is an amount obtained by integrating an amount of light of the first reflected light pulse detected in the first period.

12. The optical measuring device according to claim 9, wherein the signal processing circuit generates the information based on a fluctuation in ratio between the first signal and the second signal.

13. The optical measuring device according to claim 9, wherein when the internal state of the target of measurement is constant, a value of a ratio between the first signal and the second signal in a case where a distance between the target of measurement and the photodetector is a first distance is substantially equal to a value of the ratio in a case where the distance between the target of measurement and the photodetector is a second distance that is different from the first distance.

14. The optical measuring device according to claim 9, wherein
the target of measurement is a living organism, and
the information indicates a fluctuation in amount of blood flow of the target of measurement.

15. The optical measuring device according to claim 14, wherein the blood flow is cerebral blood flow of the living organism.

16. The optical measuring device according to claim 9, wherein
the control circuit causes the light source and the photodetector to execute a calibration operation of adjusting the first time point and the second time point,
in the calibration operation, the control circuit causes the light source to emit second light pulses and causes the photodetector to detect second reflected light pulses attributed to the second light pulses, the second reflected light pulses being detected with a very short time shift in time lag between a start of a decrease in intensity of each of the second reflected light pulse and a start of detection,
the second reflected light pulses are each detected in a period having a third time length, and
the first time length, the second time length, and the third time length are identical to one another.
